(19) United States
(12) Patent Application Publication
(10) Pub. No.: US 2026/0200915 A1
(43) Pub. Date: Jul. 16, 2026

(54) SYNTHESIS OF SULFOXIMINE COMPOUNDS HAVING A STEREOGENIC SULFUR ATOM

(71) Applicant: SYNGENTA CROP PROTECTION AG, Basel (CH)

(72) Inventors: Simon WILLIAMS, Stein (CH); Helmars SMITS, Stein (CH)

(73) Assignee: SYNGENTA CROP PROTECTION AG, Basel (CH)

(21) Appl. No.: 19/136,506

(22) PCT Filed: Dec. 7, 2023
(86) PCT No.: PCT/EP2023/084776

§ 371 Date: Jun. 6, 2025

(30) Foreign Application Priority Data
Dec. 7, 2022 (EP) 22212046.1

(51) Int. Cl. C07D 471/04; C07D 487/04

(52) U.S. Cl. CPC C07D 471/04; C07D 487/04; C07B 2200/07

(57) ABSTRACT

A process for the preparation of compound of formula (I) which process comprises: (A) stereoselectively oxidizing a sulfanyl compound of formula (II) followed by (B) stereospecific imination of a sulfinyl compound of formula (III) is disclosed wherein the substituents are as defined in claim 1.

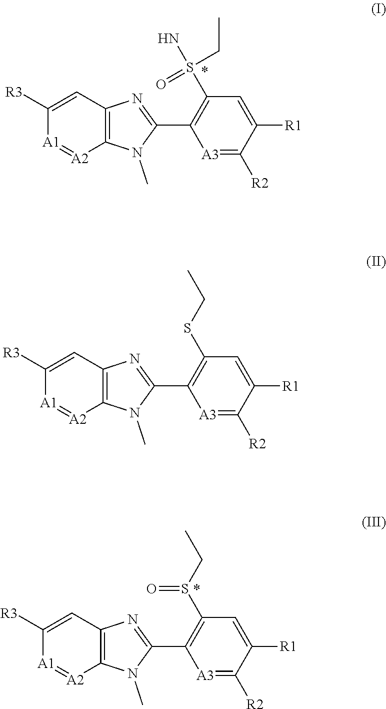

The present invention relates to the synthesis of sulfoximine compounds having a stereogenic sulfur atom.

In recent years sulfoximines have attracted great interest from the agrochemical and pharmaceutical industries as isosteres of sulfones and sulfonamides due to the ability to tune their four substituents and optimize physicochemical properties (J. Med. Chem. 2020, 63, 14243, Eur. J. Med. Chem. 2021, 209, 112885). In particular N-unsubstituted sulfoximines are of particular interest as they differ by only a single atom from a sulfone but can have significantly different properties due to the presence of a hydrogen bond donating group. Consequently, there has been a great interest in methods to introduce NH-sulfoximines and several different approaches have recently been developed as reviewed in Chem. Eur. J. 2021, 27, 17293.

Unsymmetrical sulfoximines are chiral at sulfur and therefore it is desirable to be able to prepare them in enantiopure or enantioenriched form to avoid adverse effects on human or environmental health arising from the undesired enantiomer. On small scale this can be done by separation of the enantiomers by chiral chromatography (as demonstrated in J. Med. Chem. 2021, 64, 11651), but this is an impractical and wasteful approach for large scale synthesis as the undesired enantiomer must be disposed of and high solvent volumes are often required for the separation. Methods for the enantioselective synthesis of the desired enantiomer are therefore required.

An attractive method for the synthesis of enantioenriched sulfoximines is the enantiospecific imination of an enantioenriched sulfoxide which can be prepared from the corresponding sulfide by a variety of oxidation methods (reviewed in Chem. Rev. 2020, 120, 4578, Chem. Rev. 2010, 110, 4303). Notable such methods include Kagan's titanium mediated oxidation using a tartrate ligand (J. Am. Chem. Soc. 1984, 106, 8188). A catalytic version of this protocol has been developed that avoids the use of stoichiometric titanium (Synlett. 1996, 404). Bolm has developed highly enantioselective methods using a chiral Schiff base in complex with either vanadium (Angew. Chem. Int. Ed. 1996, 34, 2640) or iron (Chem. Eur. J, 2005, 11, 1086, Angew. Chem. Int. Ed. 2004, 43, 4225) and there are related methods from Maguire using copper catalysis (J. Org. Chem. 2012, 77, 3288) or Jacobsen using manganese catalysis (Tet. Lett. 1992, 33, 7111) but these often suffer from lower selectivity. More recently List has disclosed practical organocatalytic methods that avoid metal catalysts but replace them with highly complex chiral acids (J. Am. Chem. Soc. 2012, 134, 10765, J. Am. Chem. Soc. 2021, 143, 14835). Biocatalysis using engineered enzymes offers an efficient and sustainable option for enantioselective sulfide oxidation (Catalysts, 2018, 8, 624) however the efficiency of such enzymes is highly substrate dependent and often requires extensive optimization for a single substrate.

Despite the availability of several methods for chiral sulfoxide synthesis however, the enantioselectivity and yield of many methods for enantioselective sulfoxide synthesis are very substrate dependent, particularly in the case of complex heterocyclic substrates that can often interfere with metal catalyzed reactions, and optimization of the oxidation system is often required. This need for individual optimization is exemplified by the enantioselective synthesis of esomeprazole using titanium mediated oxidation as described in Tet. Assym. 2000, 11, 3819, or iron catalysis as described in ACS Catalysis, 2018, 8, 9738. In both cases non-obvious alterations to the originally published procedures were crucial to obtain high yield and enantioselectivity on this complex substrate.

There are also various methods described for the imination of sulfoxides. Hypervalent iodine is commonly used as a reagent, either in combination with a metal catalyst such as rhodium (Org. Lett., 2004, 6, 1305), copper (Tetrahedron Lett. 1998, 39, 4805), iron (Tetrahedron Lett., 1998, 39, 5015) or silver (Org. Lett., 2005, 7, 4983) or without a catalyst as described by Bull and Luisi (Angew. Chem. Int. Ed. 2016, 51, 7203). The latter method has been used to synthesize the ATR inhibitor ceralasertib on large scale (Org. Process. Res. Dev. 2021, 25, 43), however the hypervalent iodine reagent creates significant halogenated waste and adds cost to the process. The method of Liang (Tetrahedron Lett., 2017, 58, 333-337) using NaN₃ and Eaton's reagent avoids hypervalent iodine but leads to racemization of the product when starting from an enantiopure sulfoxide starting material and is therefore inappropriate for the synthesis of chiral sulfoximines, the use of azides is also hazardous on large scale. Other alternative reagents are activated hydroxylamine derivatives such as O-mesityl-hydroxylamine (MSH) (Tetrahedron Lett., 1972, 4137, J. Org. Chem., 1974, 39, 2458). This reagent is highly unstable however and unsuitable for large scale use. Other hydroxylamine reagents have also been demonstrated such as nitrobenzolyhydroxylamine triflate in combination with an iron (Angew. Chem. Int. Ed. 2018, 57, 32) or dinitrophenylhydroxylamine in combination with a rhodium catalyst (Chem. Commun. 2014, 50, 9687).

The examples described above demonstrate that while many methods for the synthesis of chiral sulfoxides and their subsequent conversion to sulfoximines exist, it is not trivial to find a suitable method for a complex substrate that will be appropriate for large scale use.

The synthesis of various racemic sulfoximine compounds showing activity as insecticides has been described in WO2019/234158. It would be advantageous to be able to prepare these compounds in enantioenriched form as it would reduce the risk of unwanted biological, environmental or toxicological effects caused by the undesired isomer. There is no method known for the production of such sulfoximines in an enantioselective manner however. Furthermore, it would be advantageous that this method involves an enantioselective synthesis as separation of the racemic mixture by resolution and chromatography is wasteful and inefficient, particularly on large scale. Identifying such an enantioselective synthesis method is challenging however for the reasons outlined above. In particular, due to the presence of multiple heteroatoms in the substrates it would be expected that many metal catalyzed processes would be ineffective or provide poor enantioselectivity for such a substrate. It is therefore desirable to is to identify an appropriate method for the large scale, enantioselective, synthesis of these compounds that is safe, cost effective and has minimal environmental impact.

The present invention provides a process for the preparation of sulfoximines of formula (I)

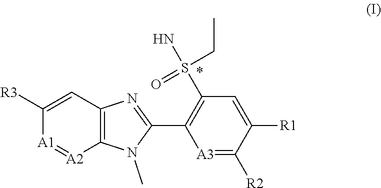

wherein
A₁, A₂ and A₃ are independently CH or N;
S* is a stereogenic sulfur atom which is in R- or S-configuration;
R₁ is cyanoisopropoxy, cyanoisopropyl or cyanocyclopropyl;
R₂ is hydrogen or methyl; and
R₃ is C₁-C₃ fluoroalkyl; which process comprises:
A) stereoselective oxidation of a sulfanyl compound of formula (II)

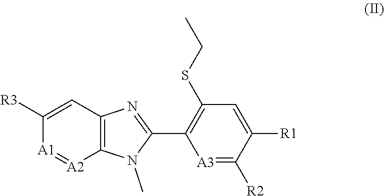

wherein A₁, A₂, A₃, R₁, R₂ and R₃ are as defined in formula (I);
in the presence of an oxidant, in the presence of a chiral catalyst, optionally in the presence of a suitable carboxylic acid or carboxylate additive, in an appropriate solvent (or diluent);
to produce a sulfinyl compound of formula (III)

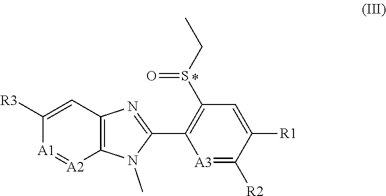

wherein A₁, A₂, A₃, R₁, R₂, R₃ and S* are as defined for compounds of formula (I);
and B) reacting the sulfinyl compound of formula (III) with an imination reagent, in the presence of a metal catalyst, optionally in the presence of a suitable acid additive, in an appropriate solvent (or diluent);
to produce the sulfoximine compound of formula (I) in a stereospecific manner.

In one embodiment, S* in formula (I) or in formula (III) is a stereogenic sulfur atom in R- or S-configuration, in which said S* center is in either enantiomerically pure or in enantiomerically enriched form.

In another embodiment, the present invention provides a process for the preparation of enantiomerically enriched sulfoximines of formula (I).

The ratio of enantiomers produced in the process can be increased by crystallization if required. Such methods are known to those skilled in the art and include crystallization from an organic solvent, a mixture of organic solvents or a mixture of organic solvents with water.

In another embodiment, the present invention provides a process for the preparation of enantiopure sulfoximines of formula (I).

Further embodiments according to the process of the invention are provided as set out below.

### Process Step (A):

In one embodiment of the invention, process step (A) comprises the stereoselective oxidation of a sulfanyl compound of formula (II)

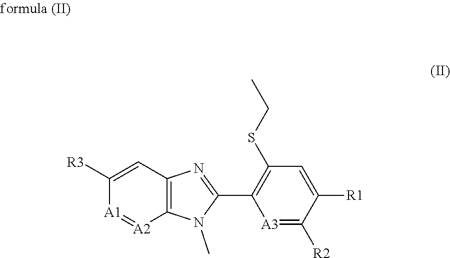

wherein

A₁, A₂ and A₃ are independently CH or N;

R₁ is cyanoisopropoxy, cyanoisopropyl or cyanocyclopropyl;

R₂ is hydrogen or methyl; and

R₃ is C₁-C₃ fluoroalkyl;

wherein the sulfanyl compound of formula (II) is oxidized, in the presence of an oxidant, in the presence of a metal derivative, in the presence of a chiral ligand, in an appropriate solvent (or diluent) and optionally in the presence of a suitable carboxylic acid or carboxylate additive to produce a sulfinyl compound of formula (III)

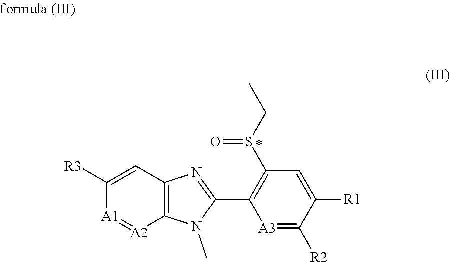

wherein A₁, A₂, A₃, R₁, R₂, and R₃ are as defined for compounds of formula (II) and S* is a stereogenic sulfur atom which is in R- or S-configuration.

Example of suitable and preferred oxidants for step A are inorganic peroxides, such as hydrogen peroxide or organic peroxides, such as tert-butyl hydroperoxide. Preferably the oxidant is hydrogen peroxide. The ratio of the oxidant used, compared to the sulfanyl compound of formula (II), is in the range from 8:1 to 0.8:1, preferably between 5:1 and 1:1, more preferably between 3:1 and 1:1.

Example of suitable and preferred metal derivatives for step A are salts of vanadium, titanium, copper, iron, manganese, molybdenum or zirconium. More preferably metal derivatives for step A are salts of vanadium or iron. Suitable examples include VOCl₂, VO(acac)₂, Fe(acac)₃, Fe(acac)₂. The amount of metal catalyst used, compared to the sulfanyl compounds of formula (III) is in the range from 0.1 mol % to 200 mol %. Preferably between 1 mol % and 10 mol %.

Examples of suitable and preferred chiral ligands for step A are derivatives of N,N′-bis(salicylidene)ethylenediamine (salen ligand) or chosen from Schiff bases formed from salicaldehyde derivatives and chiral amines.

In a preferred embodiment of the invention the metal derivative is iron or vanadium and the chiral ligand is a Schiff base formed from salicaldehyde derivatives and chiral amino-alcohols represented by a compound of formula (IV),

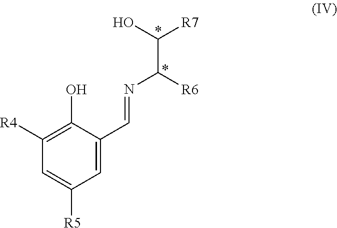

Wherein R₄ and R₅, are independently chosen from hydrogen, halogen, C₁-C₆ alkyl, C₃-C₆ cycloalkyl C₁-C₆ haloalkyl, nitro, cyano, C₁-C₄ alkoxy, C₁-C₄ haloalkoxy, optionally substituted aryl. R₆ is C₁-C₆ alkyl, optionally substituted with phenyl, 4-hydroxyphenyl, heteroaryl, hydroxy, sulfhydryl, C₁-C₆ alkoxy, C₁-C₆ alkylthio, C(O)ORₓ, C(O)NR_{Y}R_{Z}, NR_{Y}R_{Z}, guanidyl, C₃-C₆ cycloalkyl, optionally substituted aryl. Rₓ, R_{y} and R_{z} are independently selected from hydrogen and C₁-C₆ alkyl.

R₇ is hydrogen, C₁-C₆ alkyl, C₃-C₆ cycloalkyl, optionally substituted aryl or a carbonyl group (═O). R₆ and R₇ can optionally be linked to form a cyclic group. * represents (where appropriate) an enantioenriched chiral center in either R or S configuration.

Preferably R₄ and R₅ are halogen, C₁-C₄ alkyl, C₁-C₄ haloalkyl. R₆ is C₁-C₆ alkyl and R₇ is hydrogen.

Even more preferably the chiral ligand is selected from the following:
(2R)-2-[(E)-(3,5-diiodophenyl)methyleneamino]-3,3-dimethyl-butan-1-ol
(2S)-2-[(E)-(3,5-diiodophenyl)methyleneamino]-3,3-dimethyl-butan-1-ol
(2R)-2-[(E)-(3,5-dibromophenyl)methyleneamino]-3,3-dimethyl-butan-1-ol
(2S)-2-[(E)-(3,5-dibromophenyl)methyleneamino]-3,3-dimethyl-butan-1-ol
(2R)-2-[(E)-(3,5-dichlorophenyl)methyleneamino]-3,3-dimethyl-butan-1-ol
(2S)-2-[(E)-(3,5-dichlorophenyl)methyleneamino]-3,3-dimethyl-butan-1-ol
(2R)-2-[(E)-(3,5-di-tertbutyl-phenyl)methyleneamino]-3,3-dimethyl-butan-1-ol
(2S)-2-[(E)-(3,5-di-tertbutyl-phenyl)methyleneamino]-3,3-dimethyl-butan-1-ol

The chiral ligand is used as an enantioenriched compound. The enantiomeric ratio of the ligand is from 70:30 to 100:0 [R]:[S] or [S]:[R], preferably the enantiomeric ratio of the ligand is from 90:10 to 100:0 [R]:[S] or [S]:[R].

The amount of the ligand used, compared to the sulfanyl compound of formula (II), is in the range from 0.01 to 30 mol %, preferably from 1 to 15 mol %, most preferably from 2 to 10 mol %.

Optionally the ligand can be formed in situ in the reaction by adding the appropriate salicaldehyde derivative and the appropriate aminoalcohol. Alternatively, the ligand can be prepared in a separate step.

Example of suitable and preferred additives for step A are carboxylic acids. Preferably the additive is a benzoic acid, optionally mono-, di- or tri-substituted by methyl, ethyl, isopropyl, methoxy or dimethylamino, optionally in form of a lithium, sodium or potassium salt. More preferably the additive is a methoxybenzoic acid or a dimethylaminobenzoic acid (optionally in form of a lithium, sodium or potassium salt), even more preferably 4-methoxybenzoic acid. The amount of the additive used, compared to the sulfanyl compound of formula (II), is in the range from 0.01 to 10 mol %, preferably from 0.1 to 8 mol %, most preferably from 1 to 5 mol %.

In a preferred embodiment of step A the oxidizing agent is hydrogen peroxide, the metal salt is Fe(acac) 3, and the ligand is selected from:
(2R)-2-[(E)-(3,5-diiodophenyl)methyleneamino]-3,3-dimethyl-butan-1-ol,
(2S)-2-[(E)-(3,5-diiodophenyl)methyleneamino]-3,3-dimethyl-butan-1-ol,
(2R)-2-[(E)-(3,5-dibromophenyl)methyleneamino]-3,3-dimethyl-butan-1-ol,
(2S)-2-[(E)-(3,5-dibromophenyl)methyleneamino]-3,3-dimethyl-butan-1-ol,
(2R)-2-[(E)-(3,5-dichlorophenyl)methyleneamino]-3,3-dimethyl-butan-1-ol,
(2S)-2-[(E)-(3,5-dichlorophenyl)methyleneamino]-3,3-dimethyl-butan-1-ol, and the additive is 4-methoxybenzoic acid.

In another preferred embodiment of step A the oxidant is hydrogen peroxide, the metal salt is VO(acac)₂ and the ligand is (2R)-2-[(E)-(3,5-di-tertbutyl-phenyl)methyleneamino]-3,3-dimethyl-butan-1-ol or (2S)-2-[(E)-(3,5-di-tertbutyl-phenyl)methyleneamino]-3,3-dimethyl-butan-1-ol.

Examples of suitable and preferred solvents (or diluents) for step A are esters, nitriles, alcohols, ethers, and aliphatic, aromatic or halogenated hydrocarbons.

In particular, examples of suitable and preferred solvents (or diluents) for step A include: ethyl acetate, isopropyl acetate, acetonitrile, butyronitrile, ethanol, methanol, isopropanol, n-propanol, tetrahydrofuran, 2-methyl tetrahydrofuran, cyclopentylmethyl ether, t-butylmethyl ether, diethyl ether, 1,4-dioxane pentane, hexane, cyclohexane, heptane, dichloromethane, 1,2-dichloroethane, chloroform, benzene, toluene, xylene, chlorobenzene, fluorobenzene, dichlorobenzene, methoxybenzene, trifluoromethylbenzene, p-cymene, mesitylene, ethylbenzene, isopropylbenzene, or mixtures thereof.

Preferably the solvent used for process step A is an aromatic or halogenated hydrocarbon, for example: dichloromethane, 1,2-dichloroethane, chloroform, benzene, toluene, xylene, chlorobenzene, fluorobenzene, dichlorobenzene, methoxybenzene, trifluoromethylbenzene, p-cymene, mesitylene, ethylbenzene, isopropylbenzene, or mixtures thereof.

More preferably the solvent used for process step A is selected from: dichloromethane, toluene, xylene, chlorobenzene, methoxybenzene or mixtures thereof.

The reaction step (A) is advantageously carried out in a temperature range from approximately −20° C. to approximately 50° C., preferably from approximately −5° C. to approximately 30° C. In a preferred embodiment, the reaction is carried out in the range between 0° C. and 25° C.

The ratio of enantiomers produced in step A is from 50.5:49.5 to 100:0 [R]:[S] or [S]:[R]. Preferably the enantiomeric ratio of the product is from 70:30 to 100:0 [R]:[S] or [S]:[R], even more preferably 90:10 to 100:0 [R]:[S] or [S]:[R] The enantiomeric ratio of the product can be either lower or higher than the enantiomeric ratio of the chiral ligand used in the reaction.

The ratio of enantiomers produced in step A can be increased by crystallization if required. Such methods are known to those skilled in the art and include crystallization from an organic solvent, a mixture of organic solvents or a mixture of organic solvents with water.

Further preferred embodiments of process step (A) of the invention are:

### Embodiment (A1)

Table A-1 provides 12 compounds A-1.001 to A-1.012 of formula II wherein A1, A2, A3, R1, R2, and R3 are as defined in table Y.

Table A-2 provides 12 compounds A-2.001 to A-2.012 of formula III wherein A1, A2, A3, R1, R2, and R3 are as defined in table Y and S* is a stereogenic sulfur atom which is in R- or S-configuration.

TABLE Y
Substituent definitions of A1, A2, A3, R1, R2, R3

| Index | A1 | A2 | A3 | R1 | R2 | R3 |
|---|---|---|---|---|---|---|
| 1 | CH | N | N | cyanoisopropoxy | H | CF3 |
| 2 | N | N | N | cyanocyclopropyl | H | CF3 |
| 3 | N | CH | N | cyanoisopropyl | H | CF3 |
| 4 | CH | N | N | cyanoisopropoxy | CH3 | CF3 |
| 5 | CH | N | CH | cyanocyclopropyl | H | CF3 |
| 6 | N | CH | N | cyanocyclopropyl | H | CF3 |
| 7 | CH | N | N | cyanoisopropyl | H | CF3 |
| 8 | N | CH | N | cyanoisopropoxy | CH3 | CF3 |
| 9 | N | N | N | cyanoisopropoxy | H | CF3 |
| 10 | CH | N | N | cyanocyclopropyl | H | CF3 |
| 11 | N | CH | N | cyanoisopropoxy | CH3 | CF3 |
| 12 | N | CH | CH | cyanocyclopropyl | H | CF3 |

### Embodiment (A1-1)

In one aspect of embodiment (A1) of the invention, step (A) comprises the stereoselective oxidation of a sulfanyl compound of formula (II)

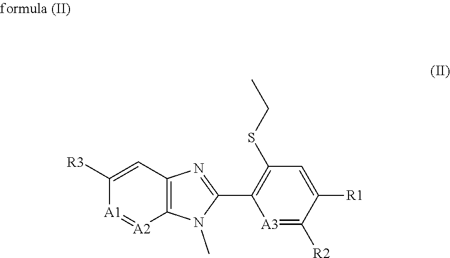

wherein
A₁, A₂ and A₃ are independently CH or N;
R₁ is cyanoisopropoxy, cyanoisopropyl or cyanocyclopropyl;
R₂ is hydrogen or methyl; and
R₃ is C₁-C₃ fluoroalkyl; which process comprises:
in the presence of an oxidant, in the presence of a metal derivative, in the presence of a chiral ligand, in an appropriate solvent (or diluent) and optionally in the presence of a suitable carboxylic acid or carboxylate additive; to produce a sulfinyl compound of formula (III);

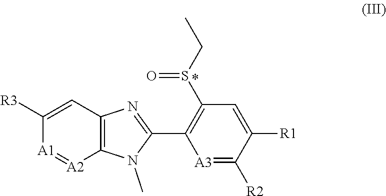

wherein A₁, A₂, A₃, R₁, R₂, and R₅ are as defined for compounds of formula (II) and S* is a stereogenic sulfur atom which is in R- or S-configuration; with the exception of a process wherein the compound of formula II is the compound A-1.001, the oxidant is hydrogen peroxide, the metal derivative is iron (III) acetylacetonate, the chiral ligand is 2-[(E)-[(1R)-1-(hydroxymethyl)-2,2-dimethyl-propyl]iminomethyl]-4,6-diiodo-phenol or is 2-[(E)-[(1S)-1-(hydroxymethyl)-2,2-dimethylpropyl]iminomethyl]-4,6-diiodo-phenol, the solvent is toluene and the carboxylic acid additive is 4-methoxybenzoic acid and the compound of formula III is the compound A-2.001.

### Embodiment (A1-2)

In another aspect of embodiment (A₁) of the invention, step (A) is conducted as described above with the exception of a process wherein the compound of formula II is the compound A-1.001, the oxidant is hydrogen peroxide (2 equivalents), the metal derivative is iron (III) acetylacetonate (5 mole %), the chiral ligand is 2-[(E)-[(1R)-1-(hydroxymethyl)-2,2-dimethyl-propyl]iminomethyl]-4,6-diiodo-phenol or is 2-[(E)-[(1S)-1-(hydroxymethyl)-2,2-dimethylpropyl]iminomethyl]-4,6-diiodo-phenol (10 mol %), the solvent is toluene and the carboxylic acid additive is 4-methoxybenzoic acid (2.5 mol %) and the compound of formula III is the compound A-2.001.

### Embodiment (A1-3)

In another aspect of embodiment (A₁) of the invention, step (A) is conducted as described above with the exception of a process wherein the compound of formula II is selected from the compound A-1.001-A-1.012, the oxidant is hydrogen peroxide (2 equivalents), the metal derivative is iron (III) acetylacetonate (5 mole %), the chiral ligand is 2-[€-[(1R)-1-(hydroxymethyl)-2,2-dimethyl-propyl]iminomethyl]-4,6-diiodo-phenol or is 2-[(E)-[(1S)-1-(hydroxymethyl)-2,2-dimethylpropyl]iminomethyl]-4,6-diiodo-phenol (10 mol %), the solvent is toluene and the carboxylic acid additive is 4-methoxybenzoic acid (2.5 mol %) and the compound of formula III is selected from the compound A-2.001-A-2.012.

### Process Step (B):

In one embodiment of the invention, process step (B) comprises the stereospecific imination of a sulfinyl compound of formula (III)

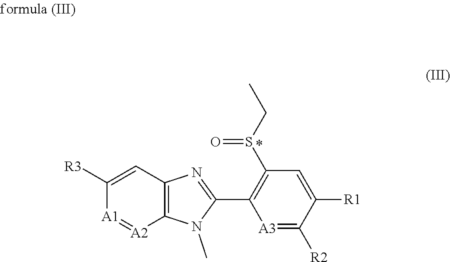

wherein
A₁, A₂ and A₃ are independently CH or N;
S* is a stereogenic sulfur atom which is in R- or S-configuration;
R₁ is cyanoisopropoxy, cyanoisopropyl or cyanocyclopropyl;
R₂ is hydrogen or methyl; and
R₃ is C₁-C₃ fluoroalkyl; which process comprises:
reacting the compound of formula (III) with an iminating reagent, in the presence of a metal catalyst, optionally in the presence of a suitable acid additive, in a suitable solvent (or diluent); to produce the sulfoximine compound of formula (I) in a stereospecific manner.

In one embodiment of the invention the imination reagent used for step B is a compound of formula (V)

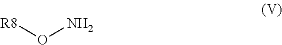

Wherein R₈ is SO₂OR₉, SO₂(R₉), C(O)R₉, P(O)(R₉)₂ or optionally substituted aryl. R₉ is Hydrogen, C₁-C₆ alkyl, C₁-C₆ haloalkyl, or optionally substituted aryl. Preferably R& is 4-nitrobenzoate, methanesulfonate, p-toluenesulfonate, hydrogensulfate, diphenylphosphinate or 2,4-dinitrophenyl.

Compounds of formula (VII) can be used as their corresponding salts represented by formula (VI)

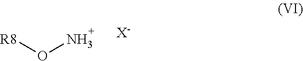

Wherein X is SO₂R₁₀, R₈ is as described for compounds of formula (VII), R₁₀ is C₁-C₆ alkyl, C₁-C₆ haloalkyl, optionally substituted aryl or OH. Preferably X is SO₂CF₃, SO₃H, or SO₂Me.

Preferably the iminating reagent is chosen from:
O-(4-nitrobenzoyl)-hydroxylamine
O-(4-nitrobenzoyl)-hydroxylammonium trifluoromethaesulfonate
O-(4-nitrobenzoyl)-hydroxylammonium hydrogensulfate
O-(4-nitrobenzoyl)-hydroxylammonium methanesulfonate
O-(2,4-dinitrophenyl)-hydroxylamine
O-(2,4-dinitrophenyl)-hydroxylammonium trifluoromethaesulfonate
O-(2,4-dinitrophenyl)-hydroxylammonium hydrogensulfate
O-(2,4-dinitrophenyl)-hydroxylammonium methanesulfonate
O-(methanesulfonyl)-hydroxylamine
O-(methanesulfonyl)-hydroxylammonium trifluoromethaesulfonate
O-(methanesulfonyl)-hydroxylammonium hydrogensulfate
O-(methanesulfonyl)-hydroxylammonium methanesulfonate
O-(p-toluenesulfonyl)-hydroxylamine
O-(p-toluenesulfonyl)-hydroxylammonium trifluoromethaesulfonate
O-(p-toluenesulfonyl)-hydroxylammonium hydrogensulfate
O-(p-toluenesulfonyl)-hydroxylammonium methanesulfonate
Hydroxylamine-O-sulfonic acid

Even more preferably the iminating reagent is chosen from:
O-(4-nitrobenzoyl)-hydroxylamine
O-(4-nitrobenzoyl)-hydroxylammonium trifluoromethaesulfonate
O-(4-nitrobenzoyl)-hydroxylammonium hydrogensulfate
O-(4-nitrobenzoyl)-hydroxylammonium methanesulfonate
Hydroxylamine-O-sulfonic acid

Optionally, imination reagents of formula (VI) that are salts can be formed in-situ by adding the appropriate acid additive (for example, trifluoromethanesulfonic acid, methanesulfonic acid, sulfuric acid) as an additive to the reaction mixture along with the iminating reagent of formula (V). Alternatively, the imination reagent of formula (VI) can be added to the reaction mixture as a preformed salt.

The amount of imination reagent used, compared to the sulfinyl compound of formula (III) is in the range of 1:1 to 8:1 preferably 1:1 to 4:1.

Suitable metal catalysts are complexes of transition metals such as iron, copper, cobalt, manganese, nickel, rhodium or ruthenium. Preferably the metal is iron. Examples of suitable and preferred catalysts include: iron (II) sulfate, iron (II) chloride, iron (III) chloride, iron (II) acetate, iron (II) trifluoromethanesulfonate, iron (II) acetylacetonate or iron (III) acetylacetonate, each in combination with either 2,2′-bipyridine or 1,10-phenanthroline, or iron(II)phthalocyanine (Fe(II) phthalocyanine, FePc). Preferably the metal catalyst is iron(II)phthalocyanine. The amount of the catalyst used, compared to the sulfinyl compound of formula (III), is in the range from 0.01 to 10 mol %, preferably from 0.1 to 8 mol %, most preferably from 1 to 5 mol %.

Suitable solvents (or diluents) used for process step B are esters, nitriles, alcohols, ethers, carboxylic acids, amides and water or mixtures thereof.

Examples of appropriate and preferred solvents used for process step B include: acetonitrile, butyronitrile, methanol, ethanol, isopropanol, 2,2,2-trifluoroethanol, hexafluoroisopropanol, dichloromethane, 1,2-dichloroethane, chloroform, ethyl acetate, isopropyl acetate, tetrahydrofuran, 1,4-dioxane, acetic acid, propanoic acid, trifluoroacetic acid, dimethyl formamide, dimethylacetamide, n-methylpyrolidinone, water or mixtures thereof.

Preferably the solvent used for process step B is acetonitrile, methanol, 2,2,2-trifluoroethanol, hexafluoroisopropanol, dichloromethane, acetic acid, water or mixtures thereof.

Most preferably the solvent (or diluent) for step B is acetonitrile, acetic acid or dichloromethane or mixtures thereof.

The reaction step (B) is advantageously carried out in a temperature range from approximately −20° C. to approximately 50° C., preferably from approximately −5° C. to approximately 30° C. In a preferred embodiment, the reaction is carried out in the range between 10° C. and 25° C.

The ratio of enantiomers produced in step B is from 50.5:49.5 to 100:0 [R]:[S] or [S]:[R].

The ratio of enantiomers produced in process step B can be increased by crystallization if required. Such methods are known to those skilled in the art and include crystallization from an organic solvent, a mixture of organic solvents or a mixture of organic solvents with water.

Further preferred embodiments of process step (B) of the invention are:

### Embodiment (B1)

Table B-1 provides 12 compounds B-1.001 to B-1.012 of formula I wherein A1, A2, A3, R1, R2, and R3 are as defined in table Z and S* is a stereogenic sulfur atom which is in R- or S-configuration.

TABLE Z
Substituent definitions of A1, A2, A3, R1, R2, R3

| Index | A1 | A2 | A3 | R1 | R2 | R3 |
|---|---|---|---|---|---|---|
| 1 | CH | N | N | cyanoisopropoxy | H | CF3 |
| 2 | N | N | N | cyanocyclopropyl | H | CF3 |
| 3 | N | CH | N | cyanoisopropyl | H | CF3 |
| 4 | CH | N | N | cyanoisopropoxy | CH3 | CF3 |
| 5 | CH | N | CH | cyanocyclopropyl | H | CF3 |
| 6 | N | CH | N | cyanocyclopropyl | H | CF3 |
| 7 | CH | N | N | cyanoisopropyl | H | CF3 |
| 8 | N | CH | N | cyanoisopropoxy | CH3 | CF3 |
| 9 | N | N | N | cyanoisopropoxy | H | CF3 |
| 10 | CH | N | N | cyanocyclopropyl | H | CF3 |
| 11 | N | CH | N | cyanoisopropoxy | CH3 | CF3 |
| 12 | N | CH | CH | cyanocyclopropyl | H | CF3 |

### Embodiment (B1-1)

In one aspect of embodiment (B1) of the invention, step (B) comprises the stereospecific imination of a sulfinyl compound of formula (III)

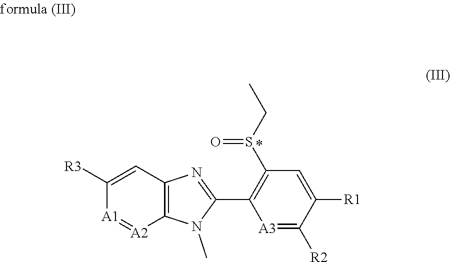

wherein
A₁, A₂ and A₃ are independently CH or N;
S* is a stereogenic sulfur atom which is in R- or S-configuration;
R₁ is cyanoisopropoxy, cyanoisopropyl or cyanocyclopropyl;
R₂ is hydrogen or methyl; and
R₃ is C₁-C₃ fluoroalkyl; which process comprises:
reacting the compound of formula (III) with an iminating reagent, in the presence of a metal catalyst, optionally in the presence of a suitable acid additive, in a suitable solvent (or diluent); to produce the sulfoximine compound of formula (I) in a stereospecific manner; with the exception of a process wherein the compound of formula III is the compound A-2.001, the iminating reagent is O-(4-nitrobenzoyl)-hydroxylamine triflic acid, the metal catalyst is iron(II)phthalocyanine, the solvent is dichloromethane and the compound of formula I is the compound B-1.001.

### Embodiment (B1-2)

In another aspect of embodiment (A₁) of the invention, step (B) is conducted as described above with the exception of a process wherein the compound of formula III is the compound A-2.001, the iminating reagent is O-(4-nitrobenzoyl)-hydroxylamine triflic acid (2 equivalents), the metal catalyst is iron(II)phthalocyanine (2 mol %), the solvent is dichloromethane and the compound of formula I is the compound B-1.001.

### Embodiment (B1-3)

In another aspect of embodiment (B1) of the invention, step (B) is conducted as described above with the exception of a process wherein the compound of formula III is selected from the compounds A-2.001-A-2.012, the iminating reagent is O-(4-nitrobenzoyl)-hydroxylamine triflic acid (2 equivalents), the metal catalyst is iron(II)phthalocyanine (2 mol %), the solvent is dichloromethane and the compound of formula I is selected from the compounds B-1.001-B-1.012.

### Definitions

The term “alkyl” as used herein, in isolation or as part of a chemical group, represents straight-chain or branched hydrocarbons, preferably with 1 to 6 carbon atoms, for example methyl, ethyl, n-propyl, isopropyl, n-butyl, isobutyl, s-butyl, t-butyl, pentyl, 1-methylbutyl, 2-methylbutyl, 3-methylbutyl, 1,2-dimethylpropyl, 1,1-dimethylpropyl, 2,2-dimethylpropyl, 1-ethylpropyl, hexyl, 1-methylpentyl, 2-methylpentyl, 3-methylpentyl, 4-methylpentyl, 1,2-dimethylpropyl, 1,3-dimethylbutyl, 1,4-dimethylbutyl, 2,3-dimethylbutyl, 1,1-dimethylbutyl, 2,2-dimethylbutyl, 3,3-dimethylbutyl, 1,1,2-trimethylpropyl, 1,2,2-tnmethylpropyl, 1-ethylbutyl and 2-ethylbutyl. Alkyl groups with 1 to 4 carbon atoms are preferred, for example methyl, ethyl, n-propyl, isopropyl, n-butyl, isobutyl, s-butyl or t-butyl.

The term “alkenyl”, in isolation or as part of a chemical group, represents straight-chain or branched hydrocarbons, preferably with 2 bis 6 carbon atoms and at least one double bond, for example vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1, 1,2-trimethyl-2-propenyl, 1-ethyl-1-methyl-2-propenyl und 1-ethyl-2-methyl-2-propenyl. Alkenyl groups with 2 to 4 carbon atoms are preferred, for example 2-propenyl, 2-butenyl or 1-methyl-2-propenyl.

The term “alkynyl”, in isolation or as part of a chemical group, represents straight-chain or branched hydrocarbons, preferably with 2 bis 6 carbon atoms and at least one triple bond, for example 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-3-butynyl, 1,2-dimethyl-3-butynyl, 2,2-dimethyl-3-butynyl, 1-ethyl-3-butynyl, 2-ethyl-3-butynyl, 1-ethyl-1-methyl-2-propynyl and 2,5-hexadiynyl Alkynyls with 2 to 4 carbon atoms are preferred, for example ethynyl, 2-propynyl or 2-butynyl-2-propenyl.

The term “cycloalkyl”, in isolation or as part of a chemical group, represents saturated or partially unsaturated mono-, bi- or tricyclic hydrocarbons, preferably 3 to 10 carbon atoms, for example cyclopropyl, cyclobutyl, cyclopentyl, cyclohexyl, cycloheptyl, cyclooctyl, bicyclo[2.2 1]heptyl, 15 bicyclo[2.2.2]octyl or adamantyl.

The term “heterocyclyl”, in isolation or as part of a chemical group-represents saturated or partially unsaturated mono-, bi- or tricyclic hydrocarbons, preferably 3 to 10 carbon atoms, which have at least one carbon atom replaced by a heteroatom selected from 0, N and S, for example tetrahydrofuran, 20 pyrrolidine, tetrahydrothiophene.

The term “aryl” represents a mono-, bi- or polycyclical aromatic system with preferably 6 to 14, more preferably 6 to 10 ring-carbon atoms, for example phenyl, naphthyl, anthryl, phenanthrenyl, preferably phenyl. “Aryl” also represents polycyclic systems, for example tetrahydronaphtyl, indenyl, indanyl, 25 fluorenyl, biphenyl. Arylalkyls are examples of substituted aryls, which may be further substituted with the same or different substituents both at the aryl or alkyl part. Benzyl and 1-phenylethyl are examples of such arylalkyls.

The term “heteroaryl” represents heteroaromatic groups, i.e. completely unsaturated aromatic 30 heterocyclic groups, which fall under the above definition of heterocycles. “Heteroaryls” with 5 to 7-membered rings with 1 to 3, preferably 1 or 2 of the same or different heteroatoms selected from N, 0, and S. Examples of “heteroaryls” are furyl, thienyl, pyrazolyl, imidazolyl, 1,2,3- and 1,2,4-triazolyl, isoxazolyl, thiazolyl, isothiazolyl, 1,2,3-, 1,3,4-, 1,2,4- and 1,2,5-oxadiazolyl, azepinyl, pyrrolyl, pyridyl, pyridazinyl, pyrimidinyl, pyrazinyl, 1,3,5-, 1,2,4- and 1,2,3-triazinyl, 1,2,4-, 1,3,2-, 1,3,6- and 1,2,6-35 oxazinyl, oxepinyl, thiepinyl, 1,2,4-triazolonyl and 1,2,4-diazepinyl.

The term “halogen” or “halo” represents fluoro, chloro, bromo or iodo, particularly fluoro, chloro or bromo. The chemical groups which are substituted with halogen, for example haloalkyl, halocycloalkyl, haloalkyloxy, haloalkylsulfanyl, haloalkylsulfinyl or haloalkylsulfonyl are substituted one or up to the maximum number of substituents with halogen. If “alkyl”, “alkenyl” or “alkynyl” are substituted with halogen, the halogen atoms can be the same or different and can be bound at the same carbon atom or different carbon atoms.

Where not otherwise defined the term “optionally substituted” means that the group in question can be substituted with zero up to the maximum number of substituents with groups independently selected from: halogen, methyl, ethyl, propyl, isopropyl, t-butyl, cyclopropyl, cyclobutyl, cyclopropyl, cyclohexyl, trifluoromethyl, difluoromethyl, chlorodifluoromethyl, trichloromethyl, methoxy, ethoxy, trifluoromethoxy, difluoromethoxy, nitro, cyano, hydroxy, sulfhydryl, acetyl, acetoxy, COOH, COOMe, COOEt, CONH₂, CONHMe, CONMe₂, amino, methlamino, dimethylamino, phenyl.

The term “enantiomerically enriched” means that one of the enantiomers of the compound is present in excess in comparison to the other enantiomer. This excess will hereafter be referred to as enantiomeric excess or ee. The ee may be determined by chiral GC, HPLC or SFC analysis. The ee is equal to the difference between amounts of enantiomers divided by the sum of the amounts of the enantiomers, which quotient can be expressed as a percentage after multiplication by 100.

In one embodiment, the process for the preparation of sulfoximines of formula (I) is carried out as described above, with the exception of a process wherein A₁ is CH, A₂ is N, A₃ is N, R₁ is cyanoisopropoxy, R₂ is H and R₃ is CF₃; in step A the oxidant is hydrogen peroxide (2 equivalents), the metal derivative is iron (III) acetylacetonate (5 mole %), the chiral ligand is 2-[(E)-[(1R)-1-(hydroxymethyl)-2,2-dimethyl-propyl]iminomethyl]-4,6-diiodo-phenol or is 2-[(E)-[(1S)-1-(hydroxymethyl)-2,2-dimethylpropyl]iminomethyl]-4,6-diiodo-phenol (10 mol %), the solvent is toluene and the carboxylic acid additive is 4-methoxybenzoic acid (2.5 mol %); and in step B the iminating reagent is O-(4-nitrobenzoyl)-hydroxylamine triflic acid (2 equivalents), the metal catalyst is iron(II)phthalocyanine (2 mol %), the solvent is dichloromethane.

In another embodiment, the process for the preparation of sulfoximines of formula (I) is carried out as described above, with the exception of a process wherein A₁, A₂, N, A₃, R₁, R₂, and R₃ are as described in table Z (indices 1-12); in step A the oxidant is hydrogen peroxide (2 equivalents), the metal derivative is iron (III) acetylacetonate (5 mole %), the chiral ligand is 2-[(E)-[(1R)-1-(hydroxymethyl)-2,2-dimethyl-propyl]iminomethyl]-4,6-diiodo-phenol or is 2-[(E)-[(1S)-1-(hydroxymethyl)-2,2-dimethylpropyl]iminomethyl]-4,6-diiodo-phenol (10 mol %), the solvent is toluene and the carboxylic acid additive is 4-methoxybenzoic acid (2.5 mol %); and in step B the iminating reagent is O-(4-nitrobenzoyl)-hydroxylamine triflic acid (2 equivalents), the metal catalyst is iron(II)phthalocyanine (2 mol %), the solvent is dichloromethane.

The following examples serve to illustrate the present invention:

TABLE 1
Synthesis of enantioenriched sulfoxides.

| Example | Structure | Condition | Result |
|---|---|---|---|
| 1-A1 SWI- 05026- 01 | 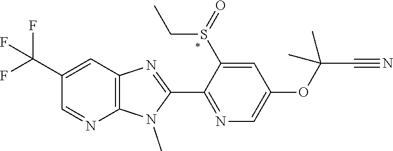 [SMILES: CCS(=O)c1cc(OC(C)(C)C#N)cnc1-c1nc2cc(C(F)(F)F)cnc2n1C] | Fe(acac)₃ 5 mol %, R- Ligand A 10 mol %, p- anisic acid 2.5 mol %, H₂O₂ (2 eq), Toluene | 73%, 85% e.e. |
| | *Enriched in enantiomer A | | |
| 1-A2 SWI- 05025- 01 | 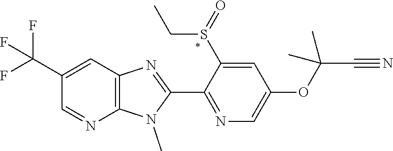 [SMILES: CCS(=O)c1cc(OC(C)(C)C#N)cnc1-c1nc2cc(C(F)(F)F)cnc2n1C] | Fe(acac)₃ 5 mol %, S- Ligand A 10 mol %, p- anisic acid 2.5 mol %, H₂O₂ (2 eq), Toluene | 87%, 88% e.e. |
| | *Enriched in enantiomer B | | |
| 1-A3 22- 46962 | 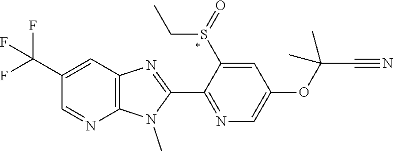 [SMILES: CCS(=O)c1cc(OC(C)(C)C#N)cnc1-c1nc2cc(C(F)(F)F)cnc2n1C] | Fe(acac)₃ 1 mol %, R- Ligand B 10 mol %, p- anisic acid 2.5 mol %, H₂O₂ (2 eq), Toluene | 70%, 76% e.e. |
| | *Enriched in enantiomer A | | |
| 1-A4 22- 46965 | 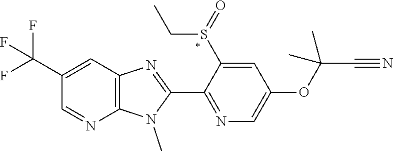 [SMILES: CCS(=O)c1cc(OC(C)(C)C#N)cnc1-c1nc2cc(C(F)(F)F)cnc2n1C] | Fe(acac)₃ 1 mol %, R- Ligand C 10 mol %, p- anisic acid 2.5 mol %, H₂O₂ (2 eq), Toluene | 73%, 88% e.e. |
| | *Enriched in enantiomer A | | |
| 2-A1 22- 47465 | 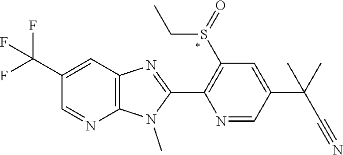 [SMILES: CCS(=O)c1cc(C(C)(C)C#N)cnc1-c1nc2cc(C(F)(F)F)cnc2n1C] | Fe(acac)₃ 5 mol %, R- Ligand B 10 mol %, p- anisic acid 2.5 mol %, H₂O₂ (2 eq), Toluene | 78% 94% e.e. |
| | Enriched in enantiomer A | | |
| 2-A2 22- 56393 | 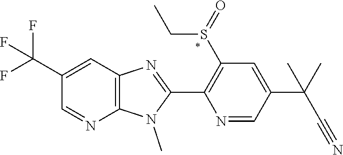 [SMILES: CCS(=O)c1cc(C(C)(C)C#N)cnc1-c1nc2cc(C(F)(F)F)cnc2n1C] | VO(acac)₂ 5 mol %, R- Ligand D 7.5 mol %, H₂O₂ (2 eq), Toluene | 36% 97% e.e. |
| | Enriched in enantiomer B | | |
| 3-A1 22- 47470 | 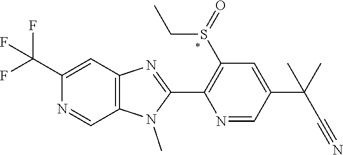 [SMILES: CCS(=O)c1cc(C(C)(C)C#N)cnc1-c1nc2cc(C(F)(F)F)ncc2n1C] | Fe(acac)₃ 5 mol %, R- Ligand C 10 mol %, p- anisic acid 2.5 mol %, H₂O₂ (2 eq), Toluene | 77%, 98% e.e. |
| | Enriched in enantiomer A | | |
| 3-A2 22- 56395 | 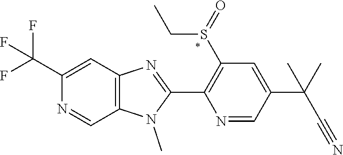 [SMILES: CCS(=O)c1cc(C(C)(C)C#N)cnc1-c1nc2cc(C(F)(F)F)ncc2n1C] | VO(acac)₂ 5 mol %, R- Ligand D 7.5 mol %, H₂O₂ (2 eq), Toluene | 45% 75% e.e. |
| | Enriched in enantiomer B | | |
| 4-A1 22- 44052 | 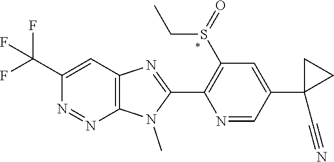 [SMILES: CCS(=O)c1cc(C2(C#N)CC2)cnc1-c1nc2cc(C(F)(F)F)nnc2n1C] | Fe(acac)₃ 5 mol %, R- Ligand A 10 mol %, p- anisic acid 2.5 mol %, H₂O₂ (2 eq), Toluene | 35%, 88% e.e. |
| | Enriched in enantiomer A | | |
| 4-A2 22- 44100 | 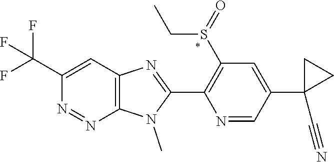 [SMILES: CCS(=O)c1cc(C2(C#N)CC2)cnc1-c1nc2cc(C(F)(F)F)nnc2n1C] | Fe(acac)₃ 5 mol %, R- Ligand A 10 mol %, p- anisic acid 2.5 mol %, H₂O₂ (2 eq), Anisole | 28%, 82% e.e. |
| | Enriched in enantiomer A | | |
| 4-A3 22- 44104 | 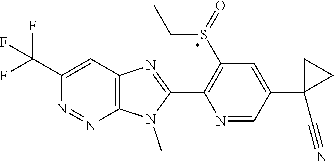 [SMILES: CCS(=O)c1cc(C2(C#N)CC2)cnc1-c1nc2cc(C(F)(F)F)nnc2n1C] | Fe(acac)₃ 1 mol %, R- Ligand A 10 mol %, p- anisic acid 2.5 mol %, H₂O₂ (2 eq), Toluene | 41%, 80% e.e. |
| | Enriched in enantiomer A | | |
| 5-A1 22- 50126 | 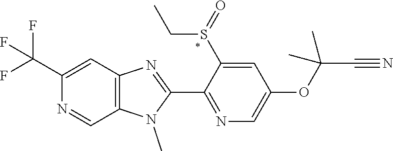 [SMILES: CCS(=O)c1cc(OC(C)(C)C#N)cnc1-c1nc2cc(C(F)(F)F)ncc2n1C] | Fe(acac)₃ 1 mol %, R- Ligand C 10 mol %, p- anisic acid 2.5 mol %, H₂O₂ (2 eq), Toluene | 78%, 88% e.e. |
| | Enriched in enantiomer A | | |
| 5-A2 22- 56394 | 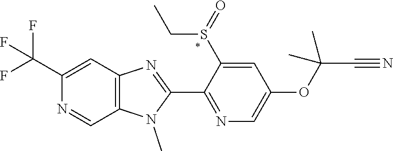 [SMILES: CCS(=O)c1cc(OC(C)(C)C#N)cnc1-c1nc2cc(C(F)(F)F)ncc2n1C] | VO(acac)₂ 5 mol %, R- Ligand D 7.5 mol %, H₂O₂ (2 eq), Toluene | 50%, 58% e.e. |
| | Enriched in enantiomer B | | |

TABLE 2
Synthesis of enantioenriched sulfoximines.

| | | | e.e. | |
|---|---|---|---|---|
| | | | of | |
| Example | Structure | Conditions | SM | Outcome |
| 1-B1 22- 56453 | 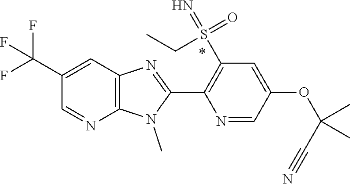 [SMILES: CCS(=N)(=O)c1cc(OC(C)(C)C#N)cnc1-c1nc2cc(C(F)(F)F)cnc2n1C] | O-(4-nitrobenzoyl)- hydroxylammonium trifluoromethaesulfonate (2.2 eq), FePc (2 mol %), CH₂Cl₂ | 85% | 81%, 83% e.e. |
| 1-B2 22- 53284 | 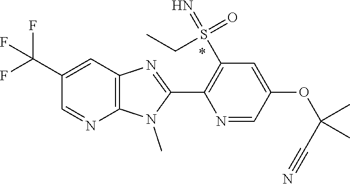 [SMILES: CCS(=N)(=O)c1cc(OC(C)(C)C#N)cnc1-c1nc2cc(C(F)(F)F)cnc2n1C] | Hydroxylamine-O- sulfonic acid (6 eq), FePc (2 mol %), AcOH | 85% | 38%, 84% e.e. |
| 2-B1 22- 51020 | 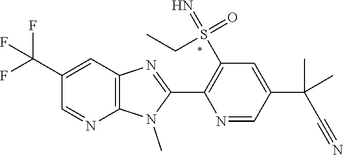 [SMILES: CCS(=N)(=O)c1cc(C(C)(C)C#N)cnc1-c1nc2cc(C(F)(F)F)cnc2n1C] | O-(4-nitrobenzoyl)- hydroxylammonium trifluoromethaesulfonate (2.2 eq), FePc (2 mol %), CH₂Cl₂ | 92% | 86%, 90% e.e. |
| 2-B2 22- 53267 | 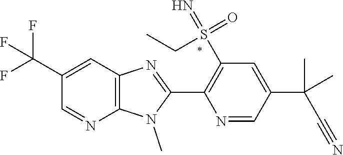 [SMILES: CCS(=N)(=O)c1cc(C(C)(C)C#N)cnc1-c1nc2cc(C(F)(F)F)cnc2n1C] | Hydroxylamine-O- sulfonic acid (6 eq), FePc (2 mol %), AcOH | 92% | 40%, 83% e.e |
| 2-B3 22- 53959 | 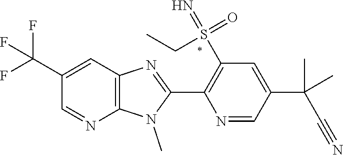 [SMILES: CCS(=N)(=O)c1cc(C(C)(C)C#N)cnc1-c1nc2cc(C(F)(F)F)cnc2n1C] | O-(4-nitrobenzoyl)- hydroxylammonium hydrogensulfate (2.5 eq), FePc (2 mol %), AcOH/MeCN | 92% | 87%, 91% e.e. |
| 3-B1 22- 51550 | 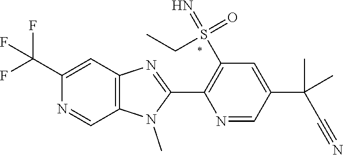 [SMILES: CCS(=N)(=O)c1cc(C(C)(C)C#N)cnc1-c1nc2cc(C(F)(F)F)ncc2n1C] | O-(4-nitrobenzoyl)- hydroxylammonium trifluoromethaesulfonate (2.2 eq), FePc (2 mol %), CH₂Cl₂ | 92% | 59%, 93% e.e. |
| 3-B2 22- 53285 | 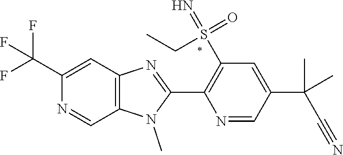 [SMILES: CCS(=N)(=O)c1cc(C(C)(C)C#N)cnc1-c1nc2cc(C(F)(F)F)ncc2n1C] | Hydroxylamine-O- sulfonic acid (6 eq), FePc (2 mol %), AcOH | 92% | 51%, 81% e.e. |
| 3-B3 22- 53281 | 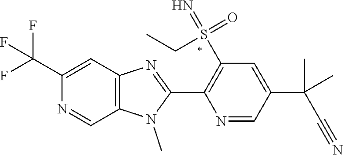 [SMILES: CCS(=N)(=O)c1cc(C(C)(C)C#N)cnc1-c1nc2cc(C(F)(F)F)ncc2n1C] | O-(4-nitrobenzoyl)- hydroxylamine (2.5 eq), methaesulfonic acid (2.5 eq), FePc (2 mol %), CH₂Cl₂ | 92% | 65%, 92% e.e |
| 4-B1 22- 51957 | 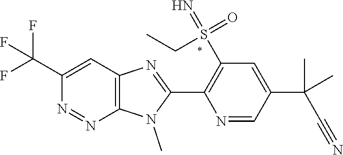 [SMILES: CCS(=N)(=O)c1cc(C(C)(C)C#N)cnc1-c1nc2cc(C(F)(F)F)nnc2n1C] | O-(4-nitrobenzoyl)- hydroxylammonium trifluoromethaesulfonate (2.2 eq), FePc (2 mol %), CH₂Cl₂ | 76% | 70%, 71% e.e. |
| 4-B2 22- 53314 | 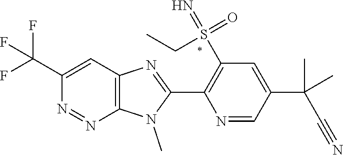 [SMILES: CCS(=N)(=O)c1cc(C(C)(C)C#N)cnc1-c1nc2cc(C(F)(F)F)nnc2n1C] | O-(2,4- Dinitrophenyl)hydroxy lamine (2.5 eq), trifluoromethanesulfonic acid (2.5 eq), FePc (2 mol %), CH₂Cl₂ | 76% | 67%, 74% e.e. |
| 5-B1 22- 51955 | 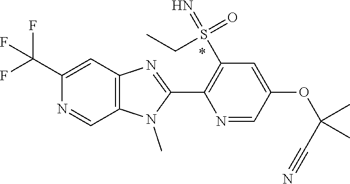 [SMILES: CCS(=N)(=O)c1cc(OC(C)(C)C#N)cnc1-c1nc2cc(C(F)(F)F)ncc2n1C] | O-(4-nitrobenzoyl)- hydroxylammonium trifluoromethaesulfonate (2.2 eq), FePc (2 mol %), CH₂Cl₂ | 93% | 75%, 89% e.e. |

### Experimental Procedures and Data

### LCMS Methods:

### Method 1

Spectra were recorded on a Mass Spectrometer from Waters (ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 □m, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: 0 min 0% B, 100% A; 1.2-1.5 min 100% B; Flow (ml/min) 0.85.

### Method 2

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 □m, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 1.2 min; Flow (ml/min) 0.85.

### Method 3

Spectra were recorded on a Mass Spectrometer from Waters (SQD, SQDII Single quadrupole mass spectrometer) equipped with an electrospray source (Polarity: positive and negative ions), Capillary: 3.00 KV, Cone range: 30V, Extractor: 2.00 V, Source Temperature: 150° C., Desolvation Temperature: 350° C., Cone Gas Flow: 50 l/h, Desolvation Gas Flow: 650 l/h, Mass range: 100 to 900 Da) and an Acquity UPLC from Waters: Binary pump, heated column compartment, diode-array detector and ELSD detector. Column: Waters UPLC HSS T3, 1.8 □m, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 2.7 min; Flow (ml/min) 0.85.

### Synthesis of Sulfide Starting Materials

### SM-1:2-[[5-ethylsulfanyl-6-[3-methyl-6-(trifluoromethyl)imidazo[4,5-b]pyridin-2-yl]-3-pyridyl]oxy]-2-methyl-propanenitrile

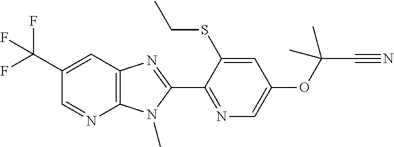 [SMILES: CCSc1cc(OC(C)(C)C#N)cnc1-c1nc2cc(C(F)(F)F)cnc2n1C]

This compound was prepared in analogy to methods described in WO2020/084075.

LCMS (method 1): m/z 422 [M+H]⁺; retention time: 1.11 min.

### SM-2:2-[5-ethylsulfanyl-6-[3-methyl-6-(trifluoromethyl)imidazo[4,5-b]pyridin-2-yl]-3-pyridyl]-2-methyl-propanenitrile

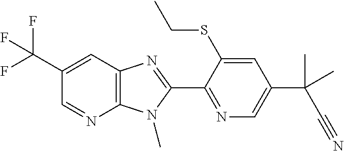 [SMILES: CCSc1cc(C(C)(C)C#N)cnc1-c1nc2cc(C(F)(F)F)cnc2n1C]

This compound was prepared in analogy to methods described in WO2018/153778.

LCMS (method 2): m/z 406 [M+H]⁺; retention time: 1.09 min.

### SM-3:2-[5-ethylsulfanyl-6-[3-methyl-6-(trifluoromethyl)imidazo[4,5-c]pyridin-2-yl]-3-pyridyl]-2-methyl-propanenitrile

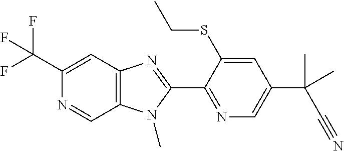 [SMILES: CCSc1cc(C(C)(C)C#N)cnc1-c1nc2cc(C(F)(F)F)ncc2n1C]

This compound was prepared in analogy to methods described in WO2018/153778 LCMS (method 2): m/z 406 [M+H]⁺; retention time: 1.02 min.

### SM-4:1-[5-ethylsulfanyl-6-[7-methyl-3-(trifluoromethyl)imidazo[4,5-c]pyridazin-6-yl]-3-pyridyl]cyclopropanecarbonitrile

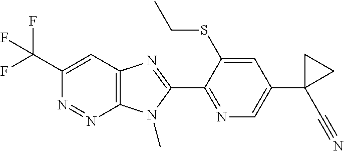 [SMILES: CCSc1cc(C2(C#N)CC2)cnc1-c1nc2cc(C(F)(F)F)nnc2n1C]

This compound was prepared in analogy to methods described in WO2019/234158.

LCMS (method 3): m/z 405 [M+H]⁺; retention time: 1.05 min.

### SM-5:2-[[5-ethylsulfanyl-6-[3-methyl-6-(trifluoromethyl)imidazo[4,5-c]pyridin-2-yl]-3-pyridyl]oxy]-2-methyl-propanenitrile

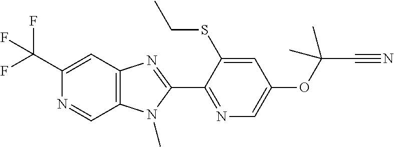 [SMILES: CCSc1cc(OC(C)(C)C#N)cnc1-c1nc2cc(C(F)(F)F)ncc2n1C]

This compound was prepared in analogy to methods described in WO2020/084075.

LCMS (method 1): m/z 422 [M+H]⁺; retention time: 1.02 min.

### Preparation of Ligands:

### Ligand (R)-A (2-[(E)-[(1R)-1-(hydroxymethyl)-2,2-dimethyl-propyl]iminomethyl]-4,6-diiodo-phenol)

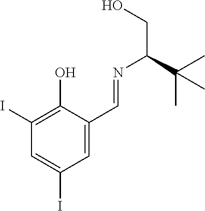 [SMILES: CC(C)(C)[C@H](CO)/N=C/c1cc(I)cc(I)c1O]

3,5-diiodosylicylaldehyde (1.12 g, 3.00 mmol) was dissolved in methanol (6.00 mL) and the resulting orange suspension was stirred at rt for 10 min before a solution of (R)-tert-leucinol (473 mg, 3.87 mmol) in methanol (6.00 mL) was added within 2 min. Stirring continued for 2 h at rt, then it was recrystallized. The reaction mixture was heated to 60° C. and stirred for 30 min until a clear solution appeared, then heating was stopped, and it was allowed to cool to 26° C. within 1.5 h. To the suspension was added drop wise 17 ml of water, the precipitate was filtered through a sintered plate and dried by evaporation to afford the title compound (1.33 g, 2.43 mmol, 81.1%).

¹H NMR (400 MHz, CDCl₃) δ (ppm)=14.07-15.38 (br s, 1H), 8.10 (s, 1H), 8.01 (d, J=1.8 Hz, 1H), 7.52 (d, J=1.8 Hz, 1H), 3.98-4.07 (dd, 1H), 3.71-3.75 (d, 1H), 3.09-3.18 (dd, 1H), 2.22-2.78 (br s, 1H), 1.02 (s, 9H).

### Ligand (S)-A (2-[(E)-[(1S)-1-(hydroxymethyl)-2,2-dimethyl-propyl]iminomethyl]-4,6-diiodo-phenol)

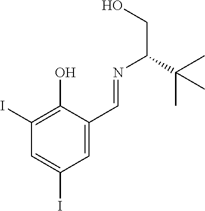 [SMILES: CC(C)(C)[C@@H](CO)/N=C/c1cc(I)cc(I)c1O]

A suspension of 2-hydroxy-3,5-diiodo-benzaldehyde (1.50 g, 4.01 mmol) in methanol (30.0 mL) was degassed with Argon, then a solution of (2S)-2-amino-3,3-dimethyl-butan-1-ol (544 mg, 4.41 mmol) in methanol (2.00 mL) was added and the reaction mixture was stirred at rt overnight. Afterwards it was concentrated under reduced pressure, dissolved in ethyl acetate, and washed three times with an aqueous solution of ammonium chloride and once with brine. The organic layer was dried over Na₂SO₄, filtered, and evaporated to afford the crude product which was triturated with few mL of hexane and diethyl ether to afford the desired compound (1.90 g, 3.80 mmol, 95.0%).

¹H NMR (400 MHz, CDCl₃) δ (ppm)=14.83 (br s, 1H), 8.08 (s, 1H), 8.00 (d, J=2.2 Hz, 1H), 7.51 (d, J=2.2 Hz, 1H), 4.03 (dd, J=11.6, 2.2 Hz, 1H), 3.71 (dd, J=11.3, 9.8 Hz, 1H), 3.12 (dd, J=9.4, 2.5 Hz, 1H), 2.88-3.08 (br s, 1H), 1.02 (s, 9H).

### Ligand B (2-[(E)-[(1R)-1-(hydroxymethyl)-2,2-dimethyl-propyl]iminomethyl]-4,6-dichloro-phenol)

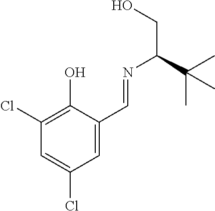 [SMILES: CC(C)(C)[C@H](CO)/N=C/c1cc(Cl)cc(Cl)c1O]

A suspension of 3,5-dichlorosalicylaldehyde (2.75 g, 13.7 mmol) in methanol (25.0 mL) was stirred at rt, then a solution of (R)-tert-leucinol (1.63 g, 13.6 mmol) in methanol (25.0 mL) was added portion wise. The solution was stirred at room temperature for 1 h, then it was heated to 60° C. for 3.5 h and stirred at 40° C. over night. The reaction mixture was evaporated to dryness and purified by flash column chromatography (silica, ethyl acetate in cyclohexane) to afford the title compound (2.68 g, 8.99 mmol, 65.8%).

¹H NMR (400 MHz, DMSO-d6) δ (ppm)=15.28 (br s, 1H), 8.51 (d, J=5.4 Hz, 1H), 7.60 (d, J=2.9 Hz, 1H), 7.54 (d, J=2.5 Hz, 1H), 4.85 (s, 1H), 3.77-3.86 (m, 1H), 3.39-3.48 (m, 1H), 3.08-3.16 (m, 1H), 0.95 (s, 9H).

### Ligand C (2-[(E)-[(1R)-1-(hydroxymethyl)-2,2-dimethyl-propyl]iminomethyl]-4,6-dibromo-phenol)

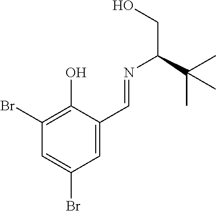 [SMILES: CC(C)(C)[C@H](CO)/N=C/c1cc(Br)cc(Br)c1O]

To a suspension of 3,5-dibromosalicylaldehyde (5.00 g, 17.5 mmol) in methanol (14.0 mL) was added a solution of (R)-tert-leucinol (2.30 g, 19.3 mmol) in methanol (3.50 mL) within 3 min. Exothermic behavior and formation of a thick suspension was observed, another portion of methanol (3.50 mL) was added. The reaction mixture was stirred at rt for 2 h, then it was filtered through a sintered plate and rinsed with several little portions of cold methanol. The filter cake was recrystallized by heating it in methanol to 60° C. and slowly cooling down to rt. The precipitate was filtered, washed with cold methanol, and evaporated to give the title compound (1.53 g, 4.04 mmol, 23.1%).

¹H NMR (400 MHz, CDCl₃) δ (ppm)=14.84 (br s, 1H), 8.13 (s, 1H), 7.61 (d, J=2.5 Hz, 1H), 7.27 (d, J=2.2 Hz, 1H), 4.02 (br s, 1H), 3.67-3.75 (m, 1H), 3.57 (br s, 1H), 3.13 (dd, J=9.4, 2.5 Hz, 1H), 1.03 (s, 9H).

### Ligand D (2-[(E)-[(1R)-1-(hydroxymethyl)-2,2-dimethyl-propyl]iminomethyl]-4,6-di-tert-butyl-phenol)

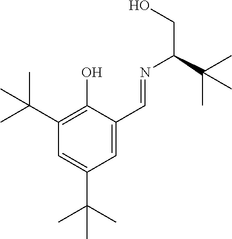 [SMILES: CC(C)(C)c1cc(/C=N/[C@@H](CO)C(C)(C)C)c(O)c(C(C)(C)C)c1]

To a solution of 3,5-di-tert-butyl-2-hydroxybenzaldehyde (2.00 g, 8.45 mmol) in methanol (84.5 mL) was added under Argon a solution of (2S)-2-amino-3,3-dimethyl-butan-1-ol (1.25 g, 10.1 mmol) in methanol (2.00 mL). The reaction mixture was heated to reflux for 1 h. After cooling down to room temperature the reaction mixture was concentrated under reduced pressure, the residue was dissolved in ethyl acetate and extracted three times with water and once with brine. The organic layer was dried over Na₂SO₄, filtered, and concentrated in vacuo to afford the title compound (3.02 g, 8.01 mmol, 94.8%).

LCMS (method 1) m/z 332 [M+H]⁻; retention time: 1.37 min.

¹H NMR (400 MHz, CDCl₃) δ ppm 13.22-14.05 (br s, 1H), 8.39 (s, 1H), 7.44 (d, J=2.5 Hz, 1H), 7.16 (d, J=2.2 Hz, 1H), 3.95 (dd, J=11.3, 2.9 Hz, 1H), 3.75-3.79 (m, 1H), 2.95 (dd, J=9.4, 2.9 Hz, 1H), 1.48 (s, 9H), 1.35 (s, 9H), 1.01 (s, 9H).

### Preparation of Racemic Sulfoxides for Development of Chiral Analytical Methods:

Racemic samples of sulfoxides are prepared according to the following general procedure: Sulfide (1 eq) is dissolved in acetic acid (5 mL/mmol) and hydrogen peroxide (1.05 equiv, 30 mass %) is added at room temperature. The reaction mixture is stirred at 40° C. for 20 h or until complete consumption of the starting material was observed by LCMS. Aqueous NaHCO₃ is added dropwise to the reaction mixture followed by ethyl acetate. The phases are separated and the aqueous phase is extracted with another portion of ethyl acetate. The combined organic layers are washed with brine, dried over MgSO₄, filtered and evaporated to afford the desired sulfoxide. The material is used directly for the development of a chiral HPLC method.

### PREPARATION OF ENANTIOENRICHED SULFOXIDES LISTED IN TABLE 1

### Example 1-A: Preparation of enantioenriched 2-[[5-ethylsulfinyl-6-[3-methyl-6-(trifluoromethyl)imidazo[4,5-b]pyridin-2-yl]-3-pyridyl]oxy]-2-methyl-propanenitrile

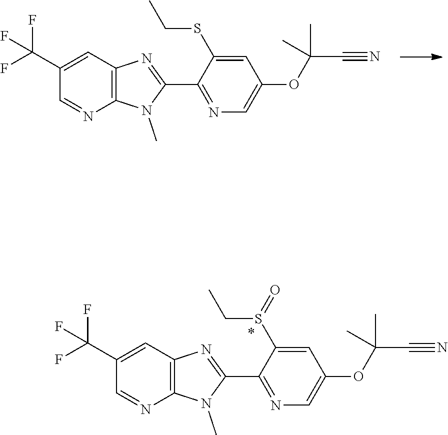 [SMILES: CCSc1cc(OC(C)(C)C#N)cnc1-c1nc2cc(C(F)(F)F)cnc2n1C ; CCS(=O)c1cc(OC(C)(C)C#N)cnc1-c1nc2cc(C(F)(F)F)cnc2n1C]

### Conditions 1-A1

2-[[5-ethylsulfanyl-6-[3-methyl-6-(trifluoromethyl)imidazo[4,5-b]pyridin-2-yl]-3-pyridyl]oxy]-2-methyl-propanenitrile (300 mg, 0.680 mmol), Iron (III) acetylacetonate (12.2 mg, 0.035 mmol), 2-[(E)-[(1R)-1-(hydroxymethyl)-2,2-dimethyl-propyl]iminomethyl]-4,6-diiodo-phenol (37.1 mg, 0.068 mmol) and 4-methoxybenzoic acid (2.6 mg, 0.017 mmol) were dissolved in toluene (2.7 mL). The solution was cooled to 0° C. and hydrogen peroxide (30% aqueous solution, 0.139 mL, 1.36 mmol) was added. The reaction was stirred at 0° C. for 30 minutes then warmed to 10° C. and stirred overnight before being warmed to rt and stirred for a further 6 h. The reaction mixture was then poured into a mixture of ethyl acetate and sodium thiosulfate solution, the layers were separated and the aqueous phase extracted with ethyl acetate. The combined organic phases were washed with water and 0.5M HCl solution, dried over sodium sulfate and concentrated in vacuo. The residue was purified by flash column chromatography (silica, ethyl acetate in cyclohexane) to afford the title compound (224 mg, 0.49 mmol, 73%)

¹H NMR (400 MHz, DMSO-d6) δ (ppm)=8.86 (d, J=1.5 Hz, 1H), 8.76 (d, J=2.5 Hz, 1H), 8.66 (d, J=1.5 Hz, 1H), 8.32 (d, J=2.9 Hz, 1H), 4.23 (s, 3H), 3.58-3.48 (m, 1H), 3.12-3.01 (m, 1H), 1.87 (apparent d, J=2.2 Hz, 6H), 1.27 (t, J=7.4 Hz, 3H)

### Chiral SFC Method

SFC: Waters Acquity UPC2/QDa
PDA Detector Waters Acquity UPC2
Column: Daicel SFC CHIRALPAK® IG, 3 μm, 0.46 cm×10 cm, 40° C.
Mobile phase: A: CO₂ B: EtOH isocratic: 20% B in 4.8 min
ABPR: 1800 psi
Flow rate: 2.0 ml/min
Detection: 310 nm
Sample concentration: 1 mg/mL in MeOH/ACN 50/50
Injection: 1 μL

### Results

| First eluting enantiomer | Second eluting enantiomer |
|---|---|
| Retention time (min) ~2.37 | Retention time (min) ~3.54 |
| Chemical purity (area % at | Chemical purity (area % at |
| 310 nm) 92.4 | 310 nm) 7.6 |

Enantiomeric excess (%) 84.8

### Conditions 1-A2

2-[[5-ethylsulfanyl-6-[3-methyl-6-(trifluoromethyl)imidazo[4,5-b]pyridin-2-yl]-3-pyridyl]oxy]-2-methyl-propanenitrile (300 mg, 0.681 mmol), Iron (III) acetylacetonate (12.3 mg, 0.035 mmol), 2-[(E)-[(1S)-1-(hydroxymethyl)-2,2-dimethyl-propyl]iminomethyl]-4,6-diiodo-phenol (35.8 mg, 0.071 mmol) and 4-methoxybenzoic acid (2.9 mg, 0.019 mmol) were dissolved in toluene (2.7 mL). The solution was cooled to 0° C. and hydrogen peroxide (30% aqueous solution, 0.139 mL, 1.36 mmol) was added. The reaction was stirred at 0° C. for 4 h then warmed to 10° C. and stirred overnight before being warmed to rt and stirred for a further 4 h. The reaction mixture was then poured into a mixture of ethyl acetate and sodium thiosulfate solution, the layers were separated, and the aqueous phase extracted with ethyl acetate. The combined organic phases were washed with water and 0.5M HCl solution, dried over sodium sulfate and concentrated in vacuo. The residue was purified by flash column chromatography (silica, ethyl acetate in cyclohexane) to afford the title compound (271 mg, 0.59 mmol, 87.3%).

NMR data as for example 1-A1

Chiral SFC method as for example 1-A1

### Results

| First eluting enantiomer | Second eluting enantiomer |
|---|---|
| Retention time (min) ~2.36 | Retention time (min) ~3.53 |
| Chemical purity (area %) 5.9 | Chemical purity (area %) 94.2 |

Enantiomeric excess (%) 88.2

### Conditions 1-A3

2-[[5-ethylsulfanyl-6-[3-methyl-6-(trifluoromethyl)imidazo[4,5-b]pyridin-2-yl]-3-pyridyl]oxy]-2-methyl-propanenitrile (200 mg, 0.456 mmol), Iron (III) acetylacetonate (1.61 mg, 0.0046 mmol), 2,4-dichloro-6-[(E)-[(1R)-1-(hydroxymethyl)-2,2-dimethyl-propyl]iminomethyl]phenol (13.5 mg, 0.046 mmol) and 4-methoxybenzoic acid (1.75 mg, 0.011 mmol) were dissolved in toluene (0.91 mL). The solution was cooled to 0° C. and hydrogen peroxide (30% aqueous solution, 0.0931 mL, 0.911 mmol) was added. The reaction was stirred at 0° C. for 4 h then warmed to rt and stirred for a further 24 h. The reaction mixture was then poured into a mixture of ethyl acetate and sodium bicarbonate solution, the layers were separated, and the aqueous phase extracted with ethyl acetate. The combined organic phases were dried over sodium sulfate and concentrated in vacuo. The residue was purified by flash column chromatography (silica, ethyl acetate in cyclohexane) to afford the title compound (146 mg, 0.32 mmol, 70.3%).

NMR data as for example 1-A1

### Chiral HPLC Method:

INSTRUMENT: Acquity UPLC from Waters
Column: Chiralpack-IG (4.6 mm×250 mm) 5 μm
Mobile phase: A: n-Hexane B: EtOH isocratic: 30% B in 45 min
Flow rate: 1.0 ml/min
Detection: 300 nm
Sample concentration: 1 mg/mL in EtOH
Injection: 5 μL

### Results

| First eluting enantiomer | Second eluting enantiomer |
|---|---|
| Retention time (min) 12.73 | Retention time (min) 23.54 |
| Chemical purity (area %) 88.2 | Chemical purity (area %) 11.8 |

Enantiomeric excess (%) 76.4%

### Conditions 1-A4

2-[[5-ethylsulfanyl-6-[3-methyl-6-(trifluoromethyl)imidazo[4,5-b]pyridin-2-yl]-3-pyridyl]oxy]-2-methyl-propanenitrile (200 mg, 0.456 mmol), Iron (III) acetylacetonate (1.61 mg, 0.0046 mmol), 2,4-dibromo-6-[(E)-[(1R)-1-(hydroxymethyl)-2,2-dimethyl-propyl]iminomethyl]phenol (17.6 mg, 0.046 mmol) and 4-methoxybenzoic acid (1.75 mg, 0.011 mmol) were dissolved in toluene (0.91 mL). The solution was cooled to 0° C. and hydrogen peroxide (30% aqueous solution, 0.0931 mL, 0.911 mmol) was added. The reaction was stirred at 0° C. for 4 h then warmed to rt and stirred for a further 24 h. The reaction mixture was then poured into a mixture of ethyl acetate and sodium bicarbonate solution, the layers were separated, and the aqueous phase extracted with ethyl acetate. The combined organic phases were dried over sodium sulfate and concentrated in vacuo. The residue was purified by flash column chromatography (silica, ethyl acetate in cyclohexane) to afford the title compound (150 mg, 0.33 mmol, 73.0%).

NMR data as for example 1-A₁

Chiral HPLC method as for example 1-A₃

### Results

| First eluting enantiomer | Second eluting enantiomer |
|---|---|
| Retention time (min) 12.79 | Retention time (min) 23.75 |
| Chemical purity (area %) 93.98 | Chemical purity (area %) 6.02 |

Enantiomeric excess (%) 88.0%

### Example 2-A Preparation of enantioenriched 2-[5-ethylsulfinyl-6-[3-methyl-6-(trifluoromethyl)imidazo[4,5-b]pyridin-2-yl]-3-pyridyl]-2-methyl-propanenitrile

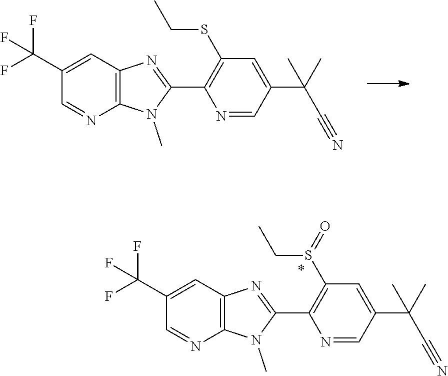 [SMILES: CCSc1cc(C(C)(C)C#N)cnc1-c1nc2cc(C(F)(F)F)cnc2n1C ; CCS(=O)c1cc(C(C)(C)C#N)cnc1-c1nc2cc(C(F)(F)F)cnc2n1C]

### Conditions 2-A1

2-[5-ethylsulfanyl-6-[3-methyl-6-(trifluoromethyl)imidazo[4,5-b]pyridin-2-yl]-3-pyridyl]-2-methyl-propanenitrile (200 mg, 0.459 mmol), Iron (III) acetylacetonate (1.62 mg, 0.0046 mmol), 2,4-dichloro-6-[(E)-[(1R)-1-(hydroxymethyl)-2,2-dimethyl-propyl]iminomethyl]phenol (13.6 mg, 0.046 mmol) and 4-methoxybenzoic acid (1.76 mg, 0.0115 mmol) were dissolved in toluene (0.92 mL). The solution was cooled to 0° C. and hydrogen peroxide (30% aqueous solution, 0.0937 mL, 0.918 mmol) was added. The reaction was stirred at rt for 19 h. The reaction mixture was then poured into a mixture of ethyl acetate and sodium bicarbonate solution, the layers were separated, and the aqueous phase extracted with ethyl acetate. The combined organic phases were dried over sodium sulfate and concentrated in vacuo. The residue was purified by flash column chromatography (silica, ethyl acetate in cyclohexane) to afford the title compound (163 mg, 0.36 mmol, 78.4%).

¹H NMR (400 MHz, DMSO-d6) δ (ppm)=9.13 (d, J=2.35 Hz, 1H), 8.88 (d, J=1.53 Hz, 1H), 8.70 (d, J=1.53 Hz, 1H), 8.61 (d, J=2.34 Hz, 1H), 4.24 (s, 3H), 3.50-3.59 (m, 1H), 2.99-3.04 (m, 1H), 1.88 (s, 3H), 1.87 (s, 3H), 1.31 (t, J=7.43 Hz, 3H).

### Chiral HPLC Method:

INSTRUMENT: Acquity UPLC from Waters
Column: Chiralpack-IG (4.6 mm×250 mm) 5 μm
Mobile phase: A: n-Hexane B: EtOH isocratic: 30% B in 40 min
Flow rate: 1.0 ml/min
Detection: 315 nm
Sample concentration: 1 mg/mL in EtOH
Injection: 5 μL

### Results

| First eluting enantiomer | Second eluting enantiomer |
|---|---|
| Retention time (min) 16.50 | Retention time (min) 25.26 |
| Chemical purity (area %) 97.43 | Chemical purity (area %) 2.57 |

Enantiomeric excess (%) 94.9%

### Conditions 2-A2

2,4-ditert-butyl-6-[(E)-[(1S)-1-(hydroxymethyl)-2,2-dimethyl-propyl]iminomethyl]phenol (30.0 mg, 0.0681 mmol) was dissolved in toluene (3.63 mL) and vanadyl acetylacetonate (12.7 mg, 0.0454 mmol) was added at rt. The mixture was stirred for 15 min, then 2-[5-ethylsulfanyl-6-[3-methyl-6-(trifluoromethyl)imidazo[4,5-b]pyridin-2-yl]-3-pyridyl]-2-methyl-propanenitrile (400 mg, 0.908 mmol) was added, followed by hydrogen peroxide (30% aqueous solution, 0.232 mL, 2.27 mmol) in four portions during 1 h. The reaction was stirred at rt for 1 h until full consumption of starting material, then it was added dropwise to a solution of sodium bicarbonate. The aqueous layer was extracted with ethyl acetate, and the combined organic phases were washed with brine, dried over sodium sulfate, and concentrated in vacuo. The residue was purified by flash column chromatography (silica, ethyl acetate in cyclohexane) to afford the title compound (139 mg, 0.33 mmol, 36.3%).

NMR data as for example 2-A₁

Chiral HPLC method as for example 2-A₁

### Results

| First eluting enantiomer | Second eluting enantiomer |
|---|---|
| Retention time (min) 16.96 | Retention time (min) 25.11 |
| Chemical purity (area %) 1.42 | Chemical purity (area %) 98.58 |

Enantiomeric excess (%) 97.16%

### Example 3-A Preparation of enantioenriched 2-[5-ethylsulfinyl-6-[3-methyl-6-(trifluoromethyl)imidazo[4,5-c]pyridin-2-yl]-3-pyridyl]-2-methyl-propanenitrile

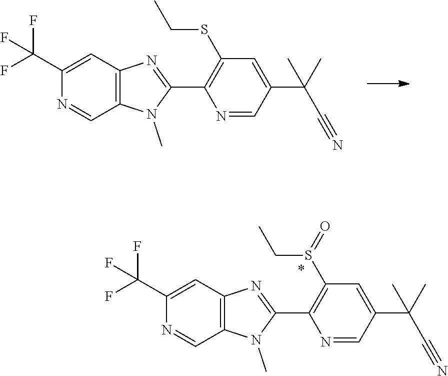 [SMILES: CCSc1cc(C(C)(C)C#N)cnc1-c1nc2cc(C(F)(F)F)ncc2n1C ; CCS(=O)c1cc(C(C)(C)C#N)cnc1-c1nc2cc(C(F)(F)F)ncc2n1C]

### Conditions 3-A1

2-[5-ethylsulfanyl-6-[3-methyl-6-(trifluoromethyl)imidazo[4,5-c]pyridin-2-yl]-3-pyridyl]-2-methyl-propanenitrile (200 mg, 0.469 mmol), Iron (III) acetylacetonate (1.66 mg, 0.0047 mmol), 2,4-dibromo-6-[(E)-[(1R)-1-(hydroxymethyl)-2,2-dimethyl-propyl]iminomethyl]phenol (18.1 mg, 0.047 mmol) and 4-methoxybenzoic acid (1.80 mg, 0.0117 mmol) were dissolved in toluene (0.94 mL). The solution was cooled to 0° C. and hydrogen peroxide (30% aqueous solution, 0.0957 mL, 0.937 mmol) was added. The reaction was stirred at rt for 19 h. The reaction mixture was then poured into a mixture of ethyl acetate and sodium bicarbonate solution, the layers were separated, and the aqueous phase extracted with ethyl acetate. The combined organic phases were dried over sodium sulfate and concentrated in vacuo. The residue was purified by flash column chromatography (silica, ethyl acetate in cyclohexane) to afford the title compound (164 mg, 0.362 mmol, 77.2%).

¹H NMR (400 MHz, DMSO-d6) δ (ppm)=9.31 (s, 1H), 9.14 (d, J=2.34 Hz, 1H), 8.61 (d, J=2.35 Hz, 1H), 8.32 (s, 1H), 4.33 (s, 3H), 3.50-3.58 (m, 1H), 3.00-3.05 (m, 1H), 1.89 (s, 3H), 1.88 (s, 3H), 1.30 (t, J=7.43 Hz, 3H)

### Chiral HPLC Method:

INSTRUMENT: Acquity UPLC from Waters
Column: Chiralpack-IG (4.6 mm×250 mm) 5 μm
Mobile phase: A: n-Hexane B: EtOH isocratic: 30% B in 40 min
Flow rate: 1.0 ml/min
Detection: 285 nm
Sample concentration: 1 mg/ml in EtOH
Injection: 5 μL

### Results

| First eluting enantiomer | Second eluting enantiomer |
|---|---|
| Retention time (min) 22.40 | Retention time (min) 26.45 |
| Chemical purity (area %) 0.88 | Chemical purity (area %) 99.12 |

Enantiomeric excess (%) 98.24%

### Conditions 3-A2

2,4-ditert-butyl-6-[(E)-[(1S)-1-(hydroxymethyl)-2,2-dimethyl-propyl]iminomethyl]phenol (57.8 mg, 0.141 mmol) was dissolved in toluene (1.87 mL) and vanadyl acetylacetonate (6.54 mg, 0.0234 mmol) was added at rt. The mixture was stirred for 15 min, then 2-[5-ethylsulfanyl-6-[3-methyl-6-(trifluoromethyl)imidazo[4,5-c]pyridin-2-yl]-3-pyridyl]-2-methyl-propanenitrile (200 mg, 0.469 mmol) was added, followed by hydrogen peroxide (30% aqueous solution, 0.0957 mL, 0.937 mmol) in one portion. The reaction was stirred at rt for 4 h until full consumption of starting material, then it was added dropwise to a solution of sodium bicarbonate. The aqueous layer was extracted with ethyl acetate, and the combined organic phases were washed with brine, dried over sodium sulfate, and concentrated in vacuo. The residue was purified by flash column chromatography (silica, ethyl acetate in cyclohexane) to afford the title compound (88 mg, 0.209 mmol, 44.6%).

NMR data as for example 3-A₁

Chiral HPLC method as for example 3-A1

### Results

| First eluting enantiomer | Second eluting enantiomer |
|---|---|
| Retention time (min) 23.75 | Retention time (min) 28.87 |
| Chemical purity (area %) 87.66 | Chemical purity (area %) 12.34 |

Enantiomeric excess (%) 75.32%

### Example 4-A Preparation of enantioenriched 1-[5-ethylsulfinyl-6-[7-methyl-3-(trifluoromethyl)imidazo[4,5-c]pyridazin-6-yl]-3-pyridyl]cyclopropanecarbonitrile

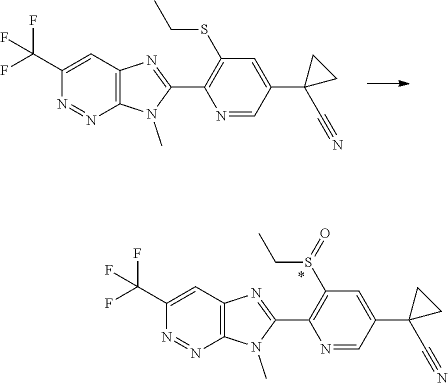 [SMILES: CCSc1cc(C2(C#N)CC2)cnc1-c1nc2cc(C(F)(F)F)nnc2n1C ; CCS(=O)c1cc(C2(C#N)CC2)cnc1-c1nc2cc(C(F)(F)F)nnc2n1C]

### Conditions 4-A1

1-[5-ethylsulfanyl-6-[7-methyl-3-(trifluoromethyl)imidazo[4,5-c]pyridazin-6-yl]-3-pyridyl]cyclopropanecarbonitrile (200 mg, 0.470 mmol), Iron (III) acetylacetonate (8.30 mg, 0.0235 mmol), 2-[(E)-[(1R)-1-(hydroxymethyl)-2,2-dimethyl-propyl]iminomethyl]-4,6-diiodo-phenol (22.5 mg, 0.047 mmol) and 4-methoxybenzoic acid (1.81 mg, 0.0117 mmol) were dissolved in toluene (0.94 mL). The solution was cooled to 0° C. and hydrogen peroxide (30% aqueous solution, 0.0960 mL, 0.940 mmol) was added. The reaction was stirred at 0° C. for 5 h and at rt for further 15 h. Since there was no full consumption of starting material the reaction mixture was heated at 40° C. for 21 h in addition. After cooling down to rt the reaction mixture was poured into a mixture of ethyl acetate and sodium bicarbonate solution, the layers were separated, and the aqueous phase extracted with ethyl acetate. The combined organic phases were dried over sodium sulfate and concentrated in vacuo. The residue was purified by flash column chromatography (silica, ethyl acetate in cyclohexane) to afford the title compound (73.0 mg, 0.160 mmol, 35.0%).

¹H NMR (400 MHz, Chloroform-d) δ (ppm)=9.09 (d, J=2.32 HZ, 1H), 8.30 (d, J=2.20 Hz, 1H), 8.20 (s, 1H), 4.57 (s, 3H), 3.57-3.69 (m, 1H), 2.97-3.0 (m, 1H), 1.97-2.08 (m, 1H), 1.92-2.14 (m, 1H), 1.63-1.79 (m, 2H), 1.45 (t, J=7.40 HZ, 3H).

### Chiral HPLC Method:

INSTRUMENT: Acquity UPLC from Waters
Column: Chiralpack-IC (4.6 mm×250 mm) 5 μm
Mobile phase: A: MTBE B: EtOH-DEA isocratic: 10% B in 30 min
Flow rate: 1.0 ml/min
Detection: 300 nm
Sample concentration: 1 mg/ml in EtOH
Injection: 5 μL

### Results

| First eluting enantiomer | Second eluting enantiomer |
|---|---|
| Retention time (min) 15.44 | Retention time (min) 21.39 |
| Chemical purity (area %) 6.13 | Chemical purity (area %) 93.87 |

Enantiomeric excess (%) 87.74%

### Conditions 4-A2

1-[5-ethylsulfanyl-6-[7-methyl-3-(trifluoromethyl)imidazo[4,5-c]pyridazin-6-yl]-3-pyridyl]cyclopropanecarbonitrile (200 mg, 0.470 mmol), Iron (III) acetylacetonate (8.30 mg, 0.0235 mmol), 2-[(E)-[(1R)-1-(hydroxymethyl)-2,2-dimethyl-propyl]iminomethyl]-4,6-diiodo-phenol (22.5 mg, 0.047 mmol) and 4-methoxybenzoic acid (1.81 mg, 0.0117 mmol) were dissolved in methoxybenzene (0.94 mL). The solution was cooled to 0° C. and hydrogen peroxide (30% aqueous solution, 0.0960 mL, 0.940 mmol) was added. The reaction was stirred at 0° C. for 5 h and at rt for further 15 h. Since there was no full consumption of starting material the reaction mixture was heated at 40° C. for 21 h in addition. After cooling down to rt the reaction mixture was poured into a mixture of ethyl acetate and sodium bicarbonate solution, the layers were separated, and the aqueous phase extracted with ethyl acetate. The combined organic phases were dried over sodium sulfate and concentrated in vacuo. The residue was purified by flash column chromatography (silica, ethyl acetate in cyclohexane) to afford the title compound (56.0 mg, 0.130 mmol, 28.0%).

NMR data as for example 4-A₁

Chiral HPLC method as for example 4-A1

### Results

| First eluting enantiomer | Second eluting enantiomer |
|---|---|
| Retention time (min) 15.44 | Retention time (min) 21.44 |
| Chemical purity (area %) 9.21 | Chemical purity (area %) 90.79 |

Enantiomeric excess (%) 81.58

### Conditions 4-A3

1-[5-ethylsulfanyl-6-[7-methyl-3-(trifluoromethyl)imidazo[4,5-c]pyridazin-6-yl]-3-pyridyl]cyclopropanecarbonitrile (200 mg, 0.470 mmol), Iron (III) acetylacetonate (1.66 mg, 0.00470 mmol), 2-[(E)-[(1R)-1-(hydroxymethyl)-2,2-dimethyl-propyl]iminomethyl]-4,6-diiodo-phenol (22.5 mg, 0.047 mmol) and 4-methoxybenzoic acid (1.81 mg, 0.0117 mmol) were dissolved in toluene (0.94 mL). The solution was cooled to 0° C. and hydrogen peroxide (30% aqueous solution, 0.0960 mL, 0.940 mmol) was added. The reaction was stirred at 0° C. for 5 h and at rt for further 15 h. Since there was no full consumption of starting material the reaction mixture was heated at 40° C. for 21 h in addition. After cooling down to rt the reaction mixture was poured into a mixture of ethyl acetate and sodium bicarbonate solution, the layers were separated, and the aqueous phase extracted with ethyl acetate. The combined organic phases were dried over sodium sulfate and concentrated in vacuo. The residue was purified by flash column chromatography (silica, ethyl acetate in cyclohexane) to afford the title compound (85.0 mg, 0.190 mmol, 41.0%).

NMR data as for example 4-A₁

Chiral HPLC method as for example 4-A1

### Results

| First eluting enantiomer | Second eluting enantiomer |
|---|---|
| Retention time (min) 15.46 | Retention time (min) 21.45 |
| Chemical purity (area %) 10.19 | Chemical purity (area %) 89.81 |

Enantiomeric excess (%) 79.62

### Example 5-A Preparation of enantioenriched 2-[[5-ethylsulfinyl-6-[3-methyl-6-(trifluoromethyl)imidazo[4,5-c]pyridin-2-yl]-3-pyridyl]oxy]-2-methyl-propanenitrile

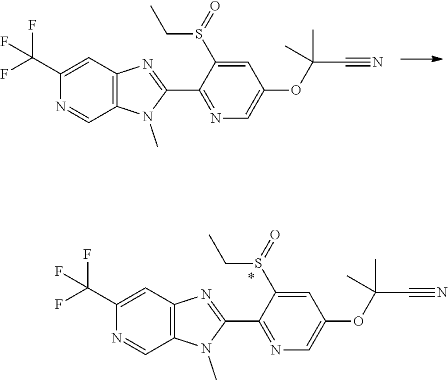 [SMILES: CCS(=O)c1cc(OC(C)(C)C#N)cnc1-c1nc2cc(C(F)(F)F)ncc2n1C ; CCS(=O)c1cc(OC(C)(C)C#N)cnc1-c1nc2cc(C(F)(F)F)ncc2n1C]

### Conditions 5-A1

2-[[5-ethylsulfanyl-6-[3-methyl-6-(trifluoromethyl)imidazo[4,5-c]pyridin-2-yl]-3-pyridyl]oxy]-2-methyl-propanenitrile (200 mg, 0.456 mmol), Iron (III) acetylacetonate (1.6 mg, 0.005 mmol), 2,4-dibromo-6-[(E)-[(1R)-1-(hydroxymethyl)-2,2-dimethyl-propyl]iminomethyl]phenol (17.6 mg, 0.046 mmol) and 4-methoxybenzoic acid (1.8 mg, 0.011 mmol) were dissolved in toluene (0.9 mL). The solution was cooled to 0° C. and hydrogen peroxide (30% aqueous solution, 0.093 mL, 0.911 mmol) was added. The reaction was stirred at rt for 15 h, then it was poured into a mixture of ethyl acetate and sodium bicarbonate solution, the layers were separated, and the aqueous phase extracted with ethyl acetate. The combined organic phases were washed with brine, dried over sodium sulfate, and concentrated in vacuo. The residue was purified by flash column chromatography (silica, ethyl acetate in cyclohexane) to afford the title compound (166 mg, 0.357 mmol, 78.3%).

¹H NMR (400 MHz Chloroform-d) δ (ppm)=9.00 (s, 1H), 8.73 (d, J=2.69 Hz, 1H), 8.42 (d, J=2.69 Hz, 1H), 8.11 (s, 1H), 4.39 (s, 3H), 3.64-3.67 (m, 1H), 3.11-3.16 (m, 1H), 1.89 (s, 3H), 1.88 (s, 3H), 1.47 (t, J=7.46 Hz, 3H).

### Chiral HPLC Method:

INSTRUMENT: Acquity UPLC from Waters
Column: Chiralpack-IG (4.6 mm×250 mm) 5 μm
Mobile phase: A: n-Hexane B: EtOH isocratic: 30% B in 40 min
Flow rate: 1.0 ml/min
Detection: 290 nm
Sample concentration: 1 mg/ml in EtOH
Injection: 10 μL

### Results

| First eluting enantiomer | Second eluting enantiomer |
|---|---|
| Retention time (min) 18.78 | Retention time (min) 22.37 |
| Chemical purity (area %) 94.10 | Chemical purity (area %) 5.90 |

Enantiomeric excess (%) 88.2

### Conditions 5-A2

2,4-ditert-butyl-6-[(E)-[(1S)-1-(hydroxymethyl)-2,2-dimethyl-propyl]iminomethyl]phenol (56.2 mg, 0.137 mmol) was dissolved in toluene (1.82 mL) and vanadyl acetylacetonate (6.36 mg, 0.0228 mmol) was added at rt. The reaction mixture was stirred at rt for 15 min, then 2-[[5-ethylsulfanyl-6-[3-methyl-6-(trifluoromethyl)imidazo[4,5-c]pyridin-2-yl]-3-pyridyl]oxy]-2-methyl-propanenitrile (200 mg, 0.456 mmol) was added, followed by hydrogen peroxide (30% aqueous solution, 0.0931 mL, 0.911 mmol). The reaction was stirred at rt for 4 h, then it was added dropwise to a saturated sodium bicarbonate solution and extracted with ethyl acetate. The combined organic phases were dried over sodium sulfate and concentrated in vacuo. The residue was purified by flash column chromatography (silica, ethyl acetate in cyclohexane) to afford the title compound (99.0 mg, 0.226 mmol, 49.7%).

NMR data as for example 5-A1

Chiral HPLC method as for example 5-A1

### Results

| First eluting enantiomer | Second eluting enantiomer |
|---|---|
| Retention time (min) 23.06 | Retention time (min) 27.33 |
| Chemical purity (area %) 20.69 | Chemical purity (area %) 79.31 |

Enantiomeric excess (%) 58.62%

### Preparation of Racemic Sulfoximines for Development of Chiral Analytical Methods

Racemic samples of sulfoximines were prepared according to the following general procedure:

The racemic sulfoxide dissolved is in dichloromethane followed by addition of (4-nitrobenzoyl)oxyammonium trifluoromethanesulfonate (2.0 equiv.) and iron(II)phthalocyanine (0.02 eq), at room temperature. The reaction mixture is stirred for 4 hours at room temperature. Aqueous NaHCO₃ solution is added dropwise and the reaction mixture is extracted with ethyl acetate. The aqueous layer is extracted again with ethyl acetate. The combined organic layers are washed with brine, dried over MgSO₄, filtered and evaporated. The crude product is purified by chromatography on silica gel using ethyl acetate and cyclohexane to afford the pure sulfoximine. The material is used for development of a chiral HPLC method.

### Preparation of Enantioenriched Sulfoximines Listed in Table 2

### Example 1-B: Preparation of 2-[[5-(ethylsulfonimidoyl)-6-[3-methyl-6-(trifluoromethyl)imidazo[4,5-b]pyridin-2-yl]-3-pyridyl]oxy]-2-methyl-propanenitrile

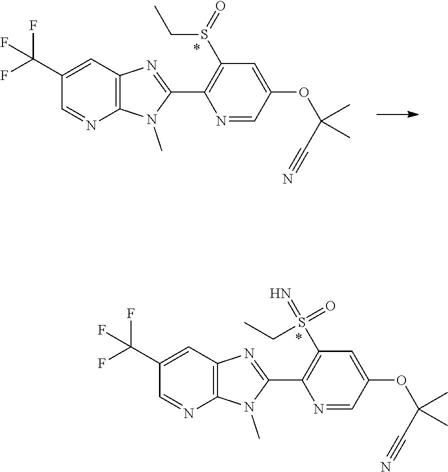 [SMILES: CCS(=O)c1cc(OC(C)(C)C#N)cnc1-c1nc2cc(C(F)(F)F)cnc2n1C ; CCS(=N)(=O)c1cc(OC(C)(C)C#N)cnc1-c1nc2cc(C(F)(F)F)cnc2n1C]

### Conditions 1-B1

2-[[5-ethylsulfinyl-6-[3-methyl-6-(trifluoromethyl)imidazo[4,5-b]pyridin-2-yl]-3-pyridyl]oxy]-2-methyl-propanenitrile (250 mg, 0.549 mmol) was dissolved in dichloromethane (1.44 ml) at room temperature under nitrogen. Iron(II)phthalocyanine (6.93 mg, 0.0110 mmol) was added, followed by addition of (4-nitrobenzoyl)oxyammonium; trifluoromethanesulfonate (446 mg, 1.21 mmol). The reaction mixture was stirred for 13 hours at room temperature then it was added dropwise to aqueous NaHCO₃ and extracted with ethyl acetate. The combined organic layers were washed with brine, dried over MgSO₄, filtered and evaporated. The crude product was purified by chromatography on silica gel using ethyl acetate and cyclohexane to afford the title compound (200 mg, 0.442 mmol, 80.6%).

¹H NMR (400 MHz, DMSO-d6) δ (ppm)=8.89 (d, J=2.57 Hz, 1H), 8.86 (d, J=1.34 Hz, 1H), 8.64 (d, J=1.83 Hz, 1H), 8.29 (d, J=2.69 Hz, 1H), 4.57 (s, 1H), 3.73 (s, 3H), 3.58-3.69 (m, 2H), 1.89 (s, 3H), 1.88 (s, 3H), 1.16 (t, J=7.34 Hz, 3H)

### Chiral HPLC Method:

INSTRUMENT: Acquity UPLC from Waters
Column: Chiralpack-IC (4.6 mm×250 mm) 5 μm
Mobile phase: A: MTBE B: EtOH isocratic: 5% B in 10 min
Flow rate: 1.0 ml/min
Detection: 290 nm
Sample concentration: 1 mg/mL in EtOH
Injection: 5 μL

### Results

| First eluting enantiomer | Second eluting enantiomer |
|---|---|
| Retention time (min) 4.69 | Retention time (min) 5.94 |
| Chemical purity (area %) 8.36 | Chemical purity (area %) 91.64 |

Enantiomeric excess (%) 83.28%

### Conditions 1-B2

2-[[5-ethylsulfinyl-6-[3-methyl-6-(trifluoromethyl)imidazo[4,5-b]pyridin-2-yl]-3-pyridyl]oxy]-2-methyl-propanenitrile (200 mg, 0.439 mmol) was dissolved in acetic acid (1.76 ml) at room temperature under nitrogen. Iron(II)phthalocyanine (5.54 mg, 0.00878 mmol) was added, followed by addition of hydroxylamine-O-sulfonic acid (205 mg, 1.76 mmol). The reaction mixture was stirred for 17 hours at room temperature then another portion of hydroxylamine-O-sulfonic acid (102 mg, 0.878 mmol) was added and stirring continued for 2 hours at room temperature. The reaction mixture was added dropwise to aqueous NaHCO₃ and extracted with ethyl acetate. The combined organic layers were washed with brine, dried over MgSO₄, filtered and evaporated. The crude product was purified by chromatography on silica gel using ethyl acetate and cyclohexane to afford the title compound (75 mg, 0.166 mmol, 37.8%).

NMR data as for example 1-B1

Chiral HPLC method as for example 1-B1

### Results

| First eluting enantiomer | Second eluting enantiomer |
|---|---|
| Retention time (min) 5.31 | Retention time (min) 7.17 |
| Chemical purity (area %) 92.11 | Chemical purity (area %) 7.89 |

Enantiomeric excess (%) 84.22%

### Example 2-B: Preparation of 2-[[5-(ethylsulfonimidoyl)-6-[3-methyl-6-(trifluoromethyl)imidazo[4,5-b]pyridin-2-yl]-3-pyridyl]-2-methyl-propanenitrile

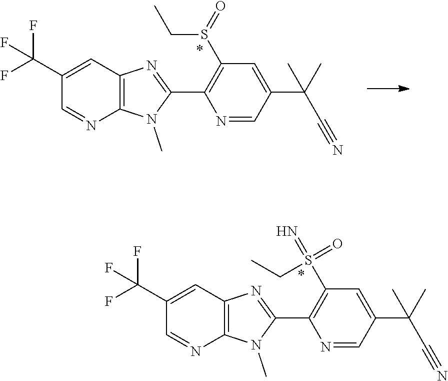 [SMILES: CCS(=O)c1cc(C(C)(C)C#N)cnc1-c1nc2cc(C(F)(F)F)cnc2n1C ; CCS(=N)(=O)c1cc(C(C)(C)C#N)cnc1-c1nc2cc(C(F)(F)F)cnc2n1C]

### Conditions 2-B1

2-[5-ethylsulfinyl-6-[3-methyl-6-(trifluoromethyl)imidazo[4,5-b]pyridin-2-yl]-3-pyridyl]-2-methyl-propanenitrile (250 mg, 0.575 mmol) was dissolved in dichloromethane (1.51 mL) at room temperature under nitrogen. Iron(II)phthalocyanine (6.61 mg, 0.0115 mmol) was added, followed by addition of (4-nitrobenzoyl)oxyammonium; trifluoromethanesulfonate (467 mg, 1.27 mmol). The reaction mixture was stirred for 13 hours at room temperature then it was added dropwise to aqueous NaHCO₃ and extracted with ethyl acetate. The combined organic layers were washed with brine, dried over MgSO₄, filtered and evaporated. The crude product was purified by chromatography on silica gel using ethyl acetate and cyclohexane to afford the title compound (216 mg, 0.495 mmol, 86.0%).

¹H NMR (400 MHz DMSO-d6) δ (ppm)=9.24 (d, J=2.20 Hz, 1H), 8.88 (d, J=1.47 Hz, 1H), 8.66 (d, J=1.83 Hz, 1H), 8.57 (d, J=2.32 Hz, 1H), 4.3 (s, 1H), 3.74 (s, 3H), 3.61-3.74 (m, 2H), 1.89 (s, 3H), 1.88 (s, 3H), 1.17-1.24 (t, 3H)

### Chiral HPLC Method:

INSTRUMENT: Acquity UPLC from Waters
Column: Chiralpack-IG (4.6 mm×250 mm) 5 μm
Mobile phase: A: n-Hexane B: EtOH isocratic: 30% B in 40 min
Flow rate: 1.0 ml/min
Detection: 290 nm
Sample concentration: 1 mg/ml in EtOH
Injection: 2 μL

### Results

| First eluting enantiomer | Second eluting enantiomer |
|---|---|
| Retention time (min) 22.15 | Retention time (min) 29.17 |
| Chemical purity (area %) 94.88 | Chemical purity (area %) 5.12 |

Enantiomeric excess (%) 89.76%

### Conditions 2-B2

2-[5-ethylsulfinyl-6-[3-methyl-6-(trifluoromethyl)imidazo[4,5-b]pyridin-2-yl]-3-pyridyl]-2-methyl-propanenitrile (200 mg, 0.460 mmol) was dissolved in acetic acid (1.84 ml) at room temperature under nitrogen. Iron(II)phthalocyanine (5.81 mg, 0.00921 mmol) was added, followed by addition of hydroxylamine-O-sulfonic acid (215 mg, 1.84 mmol). The reaction mixture was stirred for 17 hours at room temperature then another portion of hydroxylamine-O-sulfonic acid (215 mg, 1.84 mmol) was added and stirring continued for 5 hours at room temperature. The reaction mixture was added dropwise to aqueous NaHCO₃ and extracted with ethyl acetate. The combined organic layers were washed with brine, dried over MgSO₄, filtered and evaporated. The crude product was purified by chromatography on silica gel using ethyl acetate and cyclohexane to afford the title compound (80 mg, 0.183 mmol, 39.8%).

NMR data as for example 2-B1

Chiral HPLC method as for example 2-B1

### Results

| First eluting enantiomer | Second eluting enantiomer |
|---|---|
| Retention time (min) 23.97 | Retention time (min) 31.95 |
| Chemical purity (area %) 91.43 | Chemical purity (area %) 8.57 |

Enantiomeric excess (%) 82.86%

### Conditions 2-B3

2-[5-[(R)-ethylsulfinyl]-6-[3-methyl-6-(trifluoromethyl)imidazo[4,5-b]pyridin-2-yl]-3-pyridyl]-2-methyl-propanenitrile (250 mg, 0.576 mmol) was dissolved in acetonitrile (0.230 mL) and acetic acid (0.230 mL) at room temperature under nitrogen. Iron(II)phthalocyanine (7.27 mg, 0.0115 mmol) was added and it was stirred for 60 minutes at room temperature before being cooled down to 0° C. PNBHSO (417 mg, 1.44 mmol) was added and the reaction mixture was stirred for 34 hours at room temperature. Aqueous NaHCO₃ solution was added dropwise and the reaction mixture was extracted with ethyl acetate. The aqueous layer was extracted again with ethyl acetate. The combined organic layers were washed with brine, dried over Na₂SO₄, filtered and evaporated. The crude product was purified by chromatography on silica gel using ethyl acetate and cyclohexane to afford the title compound (220 mg, 0.504 mmol, 87.6%).

NMR data as for example 2-B1

Chiral HPLC method as for example 2-B1

### Results

| First eluting enantiomer | Second eluting enantiomer |
|---|---|
| Retention time (min) 22.28 | Retention time (min) 29.29 |
| Chemical purity (area %) 95.33 | Chemical purity (area %) 4.67 |

Enantiomeric excess (%) 90.66%

### Example 3-B: Preparation of 2-[[5-(ethylsulfonimidoyl)-6-[3-methyl-6-(trifluoromethyl)imidazo[4,5-c]pyridin-2-yl]-3-pyridyl]-2-methyl-propanenitrile

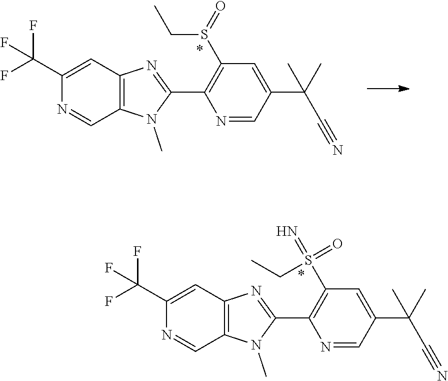 [SMILES: CCS(=O)c1cc(C(C)(C)C#N)cnc1-c1nc2cc(C(F)(F)F)ncc2n1C ; CCS(=N)(=O)c1cc(C(C)(C)C#N)cnc1-c1nc2cc(C(F)(F)F)ncc2n1C]

### Conditions 3-B1

2-[5-ethylsulfinyl-6-[3-methyl-6-(trifluoromethyl)imidazo[4,5-c]pyridin-2-yl]-3-pyridyl]-2-methyl-propanenitrile (250 mg, 0.564 mmol) was dissolved in dichloromethane (1.48 mL) at room temperature under nitrogen. Iron(II)phthalocyanine (6.47 mg, 0.0113 mmol) was added, followed by addition of (4-nitrobenzoyl)oxyammonium; trifluoromethanesulfonate (458 mg, 1.24 mmol). The reaction mixture was stirred for 13 hours at room temperature (4-then another portion of nitrobenzoyl)oxyammonium; trifluoromethanesulfonate (458 mg, 1.24 mmol) was added and stirring continued for 20 hours at room temperature. Aqueous NaHCO₃ solution was added dropwise and the reaction mixture was extracted with ethyl acetate. The combined organic layers were washed with brine, dried over Na₂SO₄, filtered and evaporated. The crude product was purified by chromatography on silica gel using ethyl acetate and cyclohexane to afford the title compound (146 mg, 0.335 mmol, 59.4%).

¹H NMR (400 MHz DMSO-d6) δ (ppm)=9.27 (s, 1H), 9.23 (d, J=2.20 Hz, 1H), 8.56 (d, J=2.20 Hz, 1H), 8.28 (s, 1H), 4.55 (s, 1H), 3.82 (s, 3H), 3.49-3.71 (m, 2H), 1.89 (s, 3H), 1.88 (s, 3H), 1.14-1.18 (t, 3H)

### Chiral HPLC Method:

INSTRUMENT: Acquity UPLC from Waters
Column: Chiralpack-IG (4.6 mm×250 mm) 5 μm
Mobile phase: A: n-Hexane B: EtOH isocratic: 30% B in 60 min
Flow rate: 1.0 ml/min
Detection: 270 nm
Sample concentration: 1 mg/ml in EtOH
Injection: 10 μL

### Results

| First eluting enantiomer | Second eluting enantiomer |
|---|---|
| Retention time (min) 31.66 | Retention time (min) 46.26 |
| Chemical purity (area %) 96.38 | Chemical purity (area %) 3.62 |

Enantiomeric excess (%) 92.76%

### Conditions 3-B2

2-[5-ethylsulfinyl-6-[3-methyl-6-(trifluoromethyl)imidazo[4,5-c]pyridin-2-yl]-3-pyridyl]-2-methyl-propanenitrile (200 mg, 0.451 mmol) was dissolved in acetic acid (1.80 ml) at room temperature under nitrogen. Iron(II)phthalocyanine (5.69 mg, 0.00902 mmol) was added, followed by addition of hydroxylamine-O-sulfonic acid (210 mg, 1.80 mmol). The reaction mixture was stirred for 17 hours at room temperature then another portion of hydroxylamine-O-sulfonic acid (105 mg, 0.902 mmol) was added and stirring continued for 2 hours at room temperature. The reaction mixture was added dropwise to aqueous NaHCO₃ and extracted with ethyl acetate. The combined organic layers were washed with brine, dried over MgSO₄, filtered and evaporated. The crude product was purified by chromatography on silica gel using ethyl acetate and cyclohexane to afford the title compound (100 mg, 0.229 mmol, 50.8%).

NMR data as for example 3-B1

Chiral HPLC method as for example 3-B1

### Results

| First eluting enantiomer | Second eluting enantiomer |
|---|---|
| Retention time (min) 31.93 | Retention time (min) 46.74 |
| Chemical purity (area %) 90.68 | Chemical purity (area %) 9.32 |

Enantiomeric excess (%) 81.36%

### Conditions 3-B3

amino 4-nitrobenzoate (261 mg, 1.41 mmol) was dissolved in dichloromethane (2.25 mL) and methanesulfonic acid (135 mg, 1.41 mmol) was added. The reaction mixture was stirred for 30 minutes at room temperature, then 2-[5-ethylsulfinyl-6-[3-methyl-6-(trifluoromethyl)imidazo[4,5-c]pyridin-2-yl]-3-pyridyl]-2-methyl-propanenitrile (250 mg, 0.564 mmol) was added, followed by Iron(II)phthalocyanine (6.41 mg, 0.0113 mmol). Stirring continued at room temperature for 32 hours, then aqueous NaHCO₃ was added dropwise and it was extracted with ethyl acetate. The combined organic layers were washed with brine, dried over Na₂SO₄, filtered and evaporated. The crude product was purified by chromatography on silica gel using ethyl acetate and cyclohexane to afford the title compound (160 mg, 0.367 mmol, 65.1%). NMR data as for example 3-B1

Chiral HPLC method as for example 3-B1

### Results

| First eluting enantiomer | Second eluting enantiomer |
|---|---|
| Retention time (min) 21.96 | Retention time (min) 30.32 |
| Chemical purity (area %) 96.21 | Chemical purity (area %) 3.79 |

Enantiomeric excess (%) 92.42%

### Example 4-B: Preparation of 1-[5-(ethylsulfonimidoyl)-6-[7-methyl-3-(trifluoromethyl)imidazo[4,5-c]pyridazin-6-yl]-3-pyridyl]cyclopropanecarbonitrile

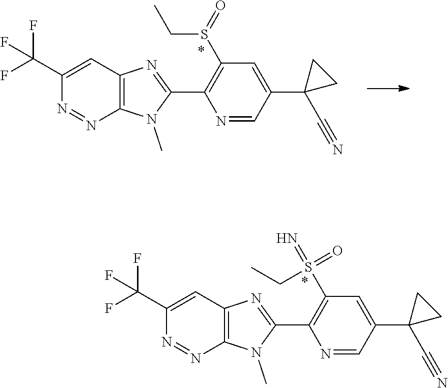 [SMILES: CCS(=O)c1cc(C2(C#N)CC2)cnc1-c1nc2cc(C(F)(F)F)nnc2n1C ; CCS(=N)(=O)c1cc(C2(C#N)CC2)cnc1-c1nc2cc(C(F)(F)F)nnc2n1C]

### Conditions 4-B1

1-[5-ethylsulfinyl-6-[7-methyl-3-(trifluoromethyl)imidazo[4,5-c]pyridazin-6-yl]-3-pyridyl]cyclopropanecarbonitrile (250 mg, 0.571 mmol) was dissolved in dichloromethane (1.50 mL) at room temperature under nitrogen. Iron(II)phthalocyanine (6.56 mg, 0.0114 mmol) was added, followed by addition of (4-nitrobenzoyl)oxyammonium; trifluoromethanesulfonate (464 mg, 1.26 mmol). The reaction mixture was stirred for 20 hours at room temperature. Aqueous NaHCO₃ solution was added dropwise and the reaction mixture was extracted with ethyl acetate. The combined organic layers were washed with brine, dried over Na₂SO₄, filtered and evaporated. The crude product was purified by chromatography on silica gel using ethyl acetate and cyclohexane to afford the title compound (173 mg, 0.397 mmol, 69.6%).

¹H NMR (400 MHz, DMSO-d6) δ (ppm)=8.96 (d, J=2.20 Hz, 1H), 8.75 (s, 1H), 8.41 (d, J=2.32 Hz, 1H), 4.64 (s, 1H), 3.87 (s, 3H), 3.48-3.65 (m, 2H), 1.88-2.07 (m, 4H), 1.15-1.18 (t, 3H).

### Chiral HPLC Method:

INSTRUMENT: Acquity UPLC from Waters
Column: Chiralpack-IC (4.6 mm×250 mm) 5 μm
Mobile phase: A: MTBE B: EtOH/DEA isocratic: 10% B in 20 min
Flow rate: 1.0 ml/min
Detection: 270 nm
Sample concentration: 1 mg/mL in EtOH
Injection: 5 μL

### Results

| First eluting enantiomer | Second eluting enantiomer |
|---|---|
| Retention time (min) 5.45 | Retention time (min) 12.32 |
| Chemical purity (area %) 14.67 | Chemical purity (area %) 85.33 |

Enantiomeric excess (%) 70.66%

### Conditions 4-B2

Amino 2,4-dinitrobenzoate (270 mg, 1.19 mmol) was dissolved in dichloromethane (1.90 mL) at room temperature under nitrogen. Trifluoromethanesulfonic acid (182 mg, 1.19 mmol) was added and the reaction mixture was stirred for 30 minutes at room temperature. 1-[5-ethylsulfinyl-6-[7-methyl-3-(trifluoromethyl)imidazo[4,5-c]pyridazin-6-yl]-3-pyridyl]cyclopropanecarbonitrile (200 mg, 0.476 mmol) was added followed by iron(II)phthalocyanine (5.41 mg, 0.00951 mmol). The reaction mixture was stirred for 24 hours at room temperature then it was added dropwise to aqueous NaHCO₃ and extracted with ethyl acetate. The combined organic layers were washed with brine, dried over Na₂SO₄, filtered and evaporated.

The crude product was purified by chromatography on silica gel using ethyl acetate and cyclohexane to afford the title compound (140 mg, 0.322 mmol, 67.6%).

NMR data as for example 4-B1

Chiral HPLC method as for example 4-B1

### Results

| First eluting enantiomer | Second eluting enantiomer |
|---|---|
| Retention time (min) 5.49 | Retention time (min) 11.91 |
| Chemical purity (area %) 13.07 | Chemical purity (area %) 86.93 |

Enantiomeric excess (%) 73.86%

### Example 5-B: Preparation of 2-[[5-(ethylsulfonimidoyl)-6-[3-methyl-6-(trifluoromethyl)imidazo[4,5-c]pyridin-2-yl]-3-pyridyl]oxy]-2-methyl-propanenitrile

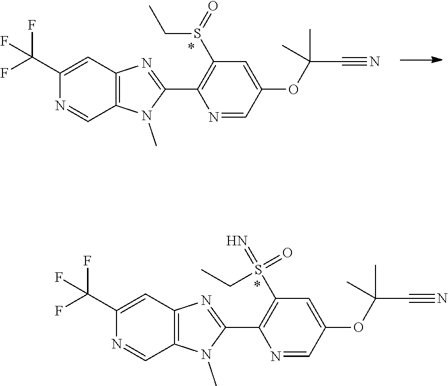 [SMILES: CCS(=O)c1cc(OC(C)(C)C#N)cnc1-c1nc2cc(C(F)(F)F)ncc2n1C ; CCS(=N)(=O)c1cc(OC(C)(C)C#N)cnc1-c1nc2cc(C(F)(F)F)ncc2n1C]

### Conditions 5-B1

2-[[5-ethylsulfinyl-6-[3-methyl-6-(trifluoromethyl)imidazo[4,5-c]pyridin-2-yl]-3-pyridyl]oxy]-2-methyl-propanenitrile (250 mg, 0.543 mmol) was dissolved in dichloromethane (1.43 mL) at room temperature under nitrogen. Iron(II)phthalocyanine (6.23 mg, 0.0109 mmol) was added, followed by addition of (4-nitrobenzoyl)oxyammonium; trifluoromethanesulfonate (441 mg, 1.19 mmol). The reaction mixture was stirred for 20 hours at room temperature then it was added dropwise to aqueous NaHCO₃ solution and extracted with ethyl acetate. The combined organic layers were washed with brine, dried over Na₂SO₄, filtered and evaporated. The crude product was purified by chromatography on silica gel using ethyl acetate and cyclohexane to afford the title compound (185 mg, 0.409 mmol, 75.3%).

¹H NMR (400 MHz, DMSO-d6) δ (ppm)=9.26 (s, 1H), 8.89 (d, J=2.69 HZ, 1H), 8.26-8.29 (m, 2H), 4.56 (s, 1H), 3.82 (s, 3H), 3.52-3.69 (m, 2H), 1.89 (s, 3H), 1.88 (s, 3H), 1.13-1.17 (t, 3H).

### Chiral HPLC Method:

INSTRUMENT: Acquity UPLC from Waters
Column: Chiralpack-IC (4.6 mm×250 mm) 5 μm
Mobile phase: A: MTBE B: EtOH/DEA isocratic: 8% B in 12 min
Flow rate: 1.0 ml/min
Detection: 274 nm
Sample concentration: 1 mg/ml in EtOH
Injection: 5 μL

### Results

| First eluting enantiomer | Second eluting enantiomer |
|---|---|
| Retention time (min) 4.34 | Retention time (min) 5.25 |
| Chemical purity (area %) 5.37 | Chemical purity (area %) 94.63 |

Enantiomeric excess (%) 89.26%

1. A process for the preparation of compound of formula (I) 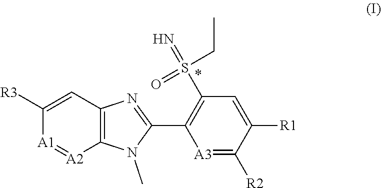
wherein
A₁, A₂ and A₃ are independently CH or N;
S* is a stereogenic sulfur atom which is in R- or S-configuration;
R₁ is cyanoisopropoxy, cyanoisopropyl or cyanocyclopropyl;
R₂ is hydrogen or methyl;
R₃ is C₁-C₃ fluoroalkyl
which process comprises:
(A) stereoselectively oxidizing a sulfanyl compound of formula (II) 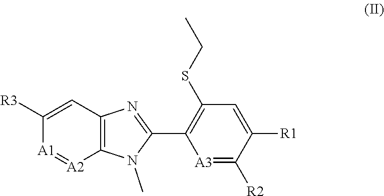
wherein A₁, A₂, A₃, R₁, R₂ and R₃ are as defined under formula (I),
in the presence of an oxidant, in the presence of a metal catalyst, in the presence of a chiral ligand, optionally in the presence of a suitable carboxylic acid or carboxylate additive additive, in an appropriate solvent (or diluent);
to produce a sulfinyl compound of formula (III) 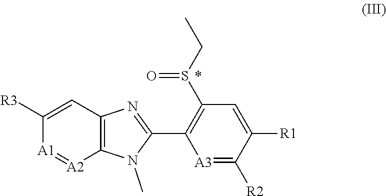
wherein A₁, A₂, A₃, R₁, R₂ and R₃ are as defined under formula (I), and
wherein S* is a stereogenic sulfur atom in R- or S-configuration; and
reacting the sulfinyl compound of formula (III) with an imination reagent, in the presence of a catalyst, optionally in the presence of a suitable acid additive, in an appropriate solvent (or diluent); to produce the sulfoximine compound of formula (I); with the exception of a process wherein A₁ is CH, A₂ is N, A₃ is N, R₁ is cyanoisopropoxy, R₂ is H and R₃ is CF₃, in step A the oxidant is hydrogen peroxide (2 equivalents), the metal derivative is iron (III) acetylacetonate (5 mole %), the chiral ligand is 2-[(E)-[(1R)-1-(hydroxymethyl)-2,2-dimethyl-propyl]iminomethyl]-4,6-diiodo-phenol or is 2-[(E)-[(1S)-1-(hydroxymethyl)-2,2-dimethylpropyl]iminomethyl]-4,6-diiodo-phenol (10 mol %), the solvent is toluene and the carboxylic acid additive is 4-methoxybenzoic acid (2.5 mol %) and in step B the iminating reagent is O-(4-nitrobenzoyl)-hydroxylamine triflic acid (2 equivalents), the metal catalyst is iron(II)phthalocyanine (2 mol %), the solvent is dichloromethane.

2. A process for the stereoselective oxidation of a sulfanyl compound of formula (II) 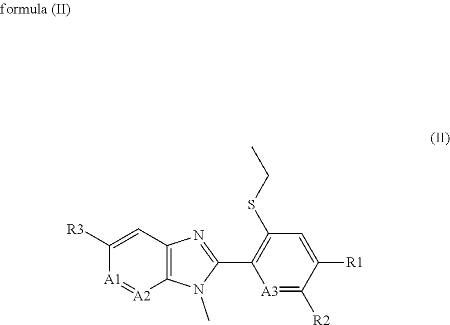
wherein
A₁, A₂ and A₃ are independently CH or N;
R₁ is cyanoisopropoxy, cyanoisopropyl or cyanocyclopropyl;
R₂ is hydrogen or methyl; and
R₃ is C₁-C₃ fluoroalkyl;
wherein the sulfanyl compound of formula (II) is oxidized, in the presence of an oxidant, in the presence of a metal derivative, in the presence of a chiral ligand, in an appropriate solvent (or diluent) and optionally in the presence of a suitable carboxylic acid or carboxylate additive to produce a sulfinyl compound of formula (III); 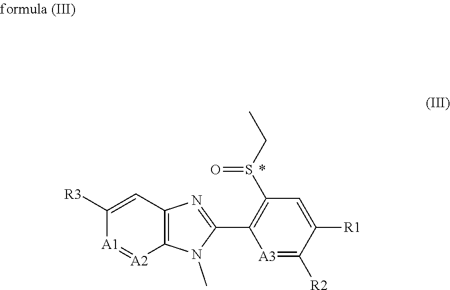
wherein A₁, A₂, A₃, R₁, R₂, and R₃ are as defined for compounds of formula (II) and S* is a stereogenic sulfur atom which is in R- or S-configuration; with the exception of a process wherein A₁ is CH, A₂ is N, A₃ is N, R₁ is cyanoisopropoxy, R₂ is H and R₃ is CF₃, in step A the oxidant is hydrogen peroxide (2 equivalents), the metal derivative is iron (III) acetylacetonate (5 mole %), the chiral ligand is 2-[(E)-[(1R)-1-(hydroxymethyl)-2,2-dimethyl-propyl]iminomethyl]-4,6-diiodo-phenol or is 2-[(E)-[(1S)-1-(hydroxymethyl)-2,2-dimethylpropyl]iminomethyl]-4,6-diiodo-phenol (10 mol %), the solvent is toluene and the carboxylic acid additive is 4-methoxybenzoic acid (2.5 mol %).

3. A process for the stereospecific imination of a sulfinyl compound of formula (III) 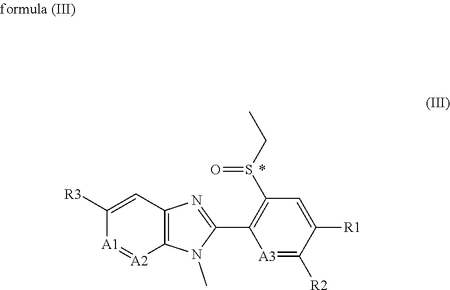
wherein
A₁, A₂ and A₃ are independently CH or N;
S* is a stereogenic sulfur atom which is in R- or S-configuration;
R₁ is cyanoisopropoxy, cyanoisopropyl or cyanocyclopropyl;
R₂ is hydrogen or methyl; and
R₃ is C₁-C₃ fluoroalkyl; which process comprises:
reacting the compound of formula (III) with an iminating reagent, in the presence of a metal catalyst, optionally in the presence of a suitable acid additive, in a suitable solvent (or diluent); to produce the sulfoximine compound of formula (I) in a stereospecific manner; 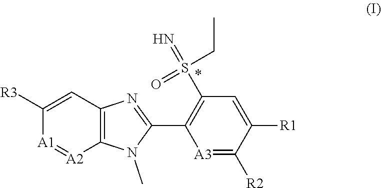
wherein A₁, A₂, A₃, R₁, R₂, R₃ and S* are as defined for compounds of formula (III); with the exception of a process wherein A₁ is CH, A₂ is N, A₃ is N, R₁ is cyanoisopropoxy, R₂ is H and R₃ is CF₃, the iminating reagent is O-(4-nitrobenzoyl)-hydroxylamine triflic acid (2 equivalents), the metal catalyst is iron(II)phthalocyanine (2 mol %), and the solvent is dichloromethane.

4. The process according to claim 1, wherein the oxidant is H₂O₂.

5. The process according to claim 1, wherein the chiral reagent is a metal complex consisting of a metal salt and a ligand of formula (IV) 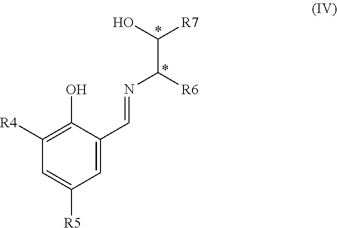
wherein R₄ and R₅, are independently chosen from hydrogen, halogen, C₁-C₆ alkyl, C₃-C₆ cycloalkyl C₁-C₆ haloalkyl, nitro, cyano, C₁-C₄ alkoxy, C₁-C₄ haloalkoxy, optionally substituted aryl. R₆ is C₁-C₆ alkyl, optionally substituted with phenyl, 4-hydroxyphenyl, heteroaryl, hydroxy, sulfhydryl, C₁-C₆ alkoxy, C₁-C₆ alkylthio, C(O)ORₓ, C(O)NR_{Y}R_{Z}, NR_{Y}R_{Z}, guanidyl, C₃-C₆ cycloalkyl, optionally substituted aryl. Rₓ, R_{y} and R_{z} are independently selected from hydrogen and C₁-C₆ alkyl; R₇ is hydrogen, C₁-C₆ alkyl, C₃-C₆ cycloalkyl, optionally substituted aryl or a carbonyl group (═O); or R₆ and R₇ can optionally be linked to form a cyclic group; and * represents (where appropriate) an enantioenriched chiral center in either R or S configuration.

6. The process according to claim 5, wherein the ligand of formula (IV) is selected from the group consisting of: (2R)-2-[(E)-(3,5-diiodophenyl)methyleneamino]-3,3-dimethyl-butan-1-ol; (2S)-2-[(E)-(3,5-diiodophenyl)methyleneamino]-3,3-dimethyl-butan-1-ol; (2R)-2-[(E)-(3,5-dibromophenyl)methyleneamino]-3,3-dimethyl-butan-1-ol; (2S)-2-[(E)-(3,5-dibromophenyl)methyleneamino]-3,3-dimethyl-butan-1-ol; (2R)-2-[(E)-(3,5-dichlorophenyl)methyleneamino]-3,3-dimethyl-butan-1-ol; (2S)-2-[(E)-(3,5-dichlorophenyl)methyleneamino]-3,3-dimethyl-butan-1-ol; (2R)-2-[(E)-(3,5-di-tertbutyl-phenyl)methyleneamino]-3,3-dimethyl-butan-1-ol; and (2S)-2-[(E)-(3,5-di-tertbutyl-phenyl)methyleneamino]-3,3-dimethyl-butan-1-ol.

7. The process according to claim 5, wherein the metal salt is an iron salt; preferably the salt is Fe(acac)₃.

8. The process according to claim 5, wherein the metal salt is a vanadium salt; preferably the salt is VO(acac)₂.

9. The process according to claim 1, wherein the carboxylic acid or carboxylic acid salt is a benzoic acid that is mono-, di- or tri-substituted by methyl, ethyl, isopropyl, methoxy or dimethylamino or is a benzoic acid that is mono-, di- or tri-substituted by methyl, ethyl, isopropyl, methoxy or dimethylamino in the form of a lithium, sodium or potassium salt salt.

10. The process according to claim 9, wherein the carboxylic acid or carboxylic acid salt is selected from methoxybenzoic acid, diaminobenzoic acid and 4-methoxybenzoic acid.

11. The process according to claim 1, wherein the imination reagent is a compound of formula (V) 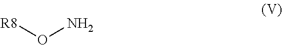
wherein R₈ is SO₂OR₉, SO₂(R₉), C(O)R₉, P(O)(R₉)₂ or optionally substituted aryl, preferably R₈ is 4-nitrobenzoate, methanesulfonate, p-toluenesulfonate, hydrogensulfate, diphenylphosphinate or 2,4-dinitrophenyl;
R₉ is hydrogen, C₁-C₆ alkyl, C₁-C₆ haloalkyl, or optionally substituted aryl, preferably R₈ is 4-nitrobenzoate, methanesulfonate, p-toluenesulfonate, hydrogensulfate, diphenylphosphinate or 2,4-dinitrophenyl;
or wherein the imination reagent is a a salt of formula (VI) 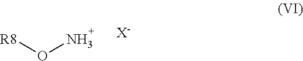
wherein X is SO₂R₁₀, R₈ is as described for compounds of formula (VII), R₁₀ is C₁-C₆ alkyl, C₁-C₆ haloalkyl, optionally substituted aryl or OH, preferably X is SO₂CF₃, SO₃H, or SO₂Me.

12. The process according to claim 11, wherein the iminating reagent is selected from: O-(4-nitrobenzoyl)-hydroxylamine; O-(4-nitrobenzoyl)-hydroxylammonium trifluoromethaesulfonate; O-(4-nitrobenzoyl)-hydroxylammonium hydrogensulfate; O-(4-nitrobenzoyl)-hydroxylammonium methanesulfonate; O-(2,4-dinitrophenyl)-hydroxylamine; O-(2,4-dinitrophenyl)-hydroxylammonium trifluoromethaesulfonate; O-(2,4-dinitrophenyl)-hydroxylammonium hydrogensulfate; O-(2,4-dinitrophenyl)-hydroxylammonium methanesulfonate; O-(methanesulfonyl)-hydroxylamine; O-(methanesulfonyl)-hydroxylammonium trifluoromethaesulfonate; O-(methanesulfonyl)-hydroxylammonium hydrogensulfate; O-(methanesulfonyl)-hydroxylammonium methanesulfonate; O-(p-toluenesulfonyl)-hydroxylamine; O-(p-toluenesulfonyl)-hydroxylammonium trifluoromethaesulfonate; O-(p-toluenesulfonyl)-hydroxylammonium hydrogensulfate; O-(p-toluenesulfonyl)-hydroxylammonium methanesulfonate; and Hydroxylamine-O-sulfonic acid.

13. The process according to claim 1, wherein the solvent (or diluent) used for step A is selected from esters, nitriles, alcohols, ethers, and aliphatic, aromatic or halogenated hydrocarbons.

14. The process according to claim 1, wherein the solvent (or diluent) used for step B is selected from esters, nitriles, alcohols, ethers, carboxylic acids, amides and water or mixtures thereof.
